(19) United States
(12) Patent Application Publication
(10) Pub. No.: US 2024/0012008 A1
(43) Pub. Date: Jan. 11, 2024

(54) COMPOSITIONS AND METHODS FOR DIAGNOSING AND TREATING PATIENTS WITH A HISTORY OF EARLY LIFE ADVERSITY

(71) Applicant: The Regents of the University of California, Oakland, CA (US)

(72) Inventors: Arpana Gupta, Los Angeles, CA (US); Emeran A. Mayer, Los Angeles, CA (US); Vadim Osadchiy, Los Angeles, CA (US)

(21) Appl. No.: 18/034,570

(22) PCT Filed: Oct. 29, 2021
(86) PCT No.: PCT/US21/57198

§ 371 Date: Apr. 28, 2023

Related U.S. Application Data
(60) Provisional application No. 63/107,998, filed on Oct. 30, 2020.

(51) Int. Cl. G01N 33/68; G01N 33/92; G01N 33/53

(52) U.S. Cl. CPC G01N 33/6893; G01N 33/6848; G01N 33/92; G01N 33/5308; G01N 2800/7042

(57) ABSTRACT

The present application relates to compositions and methods for diagnosing patients with a history of early-life adversity (ELA), and for preventing, treating, or reducing psychological distress in patients with a history of ELA.

### RELATED APPLICATION

This application claims a right of priority to and the benefit of the filing date of U.S. Provisional Application No. 63/107,998, filed on Oct. 30, 2020, which is hereby incorporated by reference in its entirety.

### GOVERNMENT SUPPORT

This invention was made with government support under Grant Numbers DK106528, DK121025, DK041301, and DK048351, awarded by the National Institutes of Health. The government has certain rights in the invention.

### BACKGROUND OF THE INVENTION

A perceived history of early-life adversity (ELA) is a known disruptor capable of inducing a range of developmental changes (1), and increases the vulnerability for a variety of conditions and psychiatric disorders later in life (2). Systemic changes in response to stress during critical periods include (dys)regulation of peripheral gene expression (3), immune function (4), and hormone levels (5), in addition to perturbations of the microbiome (6), all of which may contribute to and result from direct changes in the developing central nervous system (CNS). The involvement of the gut microbiome and its interactions with the brain during this early programming period remain incompletely understood. It has been previously proposed that this may occur in a bidirectional manner: while the brain may influence restructuring of gut physiology and microbiome composition and function, the resulting altered functional output from the gut microbiome may result in neuroplastic changes in the brain (7). Direct effects of ELA have been reported in conditions ranging from obesity (8) to irritable bowel syndrome (9-12) and inflammation (13), but few studies have used a systems approach, investigating perturbations in both the brain-gut microbiome (BGM) together in order to identify how these systems interact to produce the observed ELA relationships.

A primary pathway by which ELA can influence life-long trajectories is by shaping brain development (1). ELA is associated with alterations mainly in regions of the emotion regulation and salience networks, which in turn can influence epigenetic processes related to myelination and neurogenesis (14, 15). These neural changes have also been associated with hyperarousal and difficulties with emotion regulation, and later development of negative mood states (16-18). In particular, prefrontal cortex and hippocampal volumes were persistently reduced in adolescents adopted from international orphanages (19), and female adolescents with a history of childhood maltreatment displayed altered organization of cortical networks, which mediated psychiatric outcomes (20). Rodent research has shown similar findings with increased resolution: maternal separation was associated with accelerated innervation of basolateral amygdala axons into the prefrontal cortex, with females specifically demonstrating reduced functional connectivity between these regions across maturation, and increased anxiety-like behavior (21).

The gut microbiome is also sensitive to ELA. A number of early developmental factors have been implicated in gut microbiome development formation, especially those relating to maternal stress, diet, and disease (22), method of delivery (23, 24), early nutrition/breast-feeding (24, 25), and fetal antibiotics (23). Several animal studies have described dysbiosis following maternal separation (26) and limited bedding (27), and a robust alteration of the microbiome diversity and taxonomical profile induced by chronic social stress (28, 29). The BGM axis is a critical player in mediating normal developmental trajectories, but when faced with developmental disruptors, alterations within the BGM axis may result in negative health outcomes. For example, germ-free mice exhibit reduced anxiety-like behavior and baseline corticosterone expression (30), suggesting a bidirectional relationship between stress and the BGM axis. While some early stressors occur during the first three-year programming phase of the gut microbiome and affect the gut microbiome directly (31), subsequent gut microbial alterations may occur as a result of stress-induced alterations in the autonomic nervous system of the gut.

Microbiome signaling to the brain can be mediated by metabolite produced directly by gut microbes or indirectly from host cells responding to microbial cues (32). For example, acid and homovanillic acid—which are believed to be derived from microbial metabolism—in the cerebral spinal fluid of depressed patients were associated with neuroticism scores (33, 34), and microbiota transplanted into mice from depressed patients altered metabolite levels and behavioral outputs (35). In animal models of ELA, serotonin, the majority of which is synthesized in the gut's enterochromaffin cells, was reduced in the hypothalamus (36). Additionally, microbial metabolites such as short-chain fatty acids ameliorate cortisol induction in response to an acute psychosocial stress test in humans when delivered directly to the colon (37), and effects of early-life chronic stress in rodents when delivered orally (38), further underscoring the relationship between the BGM and stress. ELA-induced signaling pathways are capable of influencing gut bacteria and functional output, and interactions between the host and microbiome may in turn play a role in response to stressors (39). In such a way, ELA may be capable of sensitizing the body to later stressors, and one way this may manifest is via functional alterations in the gut, which then influence brain function.

While there exists a plethora of animal models relating the gut microbiome and metabolites, as well as neural regional and circuit development, to critical developmental periods and ongoing stress (6), there is a lack of comprehensive investigation of these interactions in humans. Accordingly, there is a need for human studies on relationship between early life adversity and adult psychiatric symptoms and stress, and for new diagnostic and treatment methods for patients with a history of early life adversity.

### SUMMARY OF THE INVENTION

The present invention is based, at least in part, on the discovery that history of early life adversity is associated with four gut-regulated metabolites in the glutamate (non-essential amino acid) pathway: glutamate, gamma-methyl ester, malate, urate, lithocholic acid sulfate, and 5-oxoproline. It was demonstrated herein that these metabolites were associated with perceived stress ratings in adulthood (as assessed by the validated Perceived Stress Scale questionnaire) and anxiety (as assessed by the validated Hospital Anxiety and Depression Scale-Anxiety Subset questionnaire), in addition to being associated with alterations in brain regions important for critical decision-making or negative psychological states. The observed alterations in molecules from the glutamatergic pathway may play a unique role in priming patients with a history of early life adversity to have greater sensitivity to changes in the brain that lead to more negative clinical impact such as higher stressful life events. Of note, all four of these molecules have been shown to be at least partially be microbiota-derived or microbiota-modulated. Lactobacillus plantarum, frequently found in high concentrations in fermented foods and saliva, for example, has been known to produce a large amount of glutamate and is sometimes even leveraged in industrial settings for this purpose. Additionally, microbiota enriched in Prevotella are associated with increased levels of urate.

Accordingly, in some aspects, provided herein are methods of identifying patients with a history of early-life adversity (ELA), comprising: (a) measuring the level of one or more metabolites selected from glutamate, gamma-methyl ester, 5-oxoproline, malate, lithocholic acid sulfate, and urate in a sample obtained from the subject; (b) comparing the level detected from the step (a) to the normal or control level of the metabolite; wherein a decreased level of the metabolite in the subject sample relative to the normal/control level indicates that the subject has a history of ELA.

In other aspects, provided herein are methods of preventing, treating, or reducing psychological distress in a subject with a history of ELA, comprising administering to the subject an agent that increases the level and/or activity of at least one metabolite selected from glutamate, gamma-methyl ester, 5-oxoproline, malate, lithocholic acid sulfate, and urate.

### BRIEF DESCRIPTION OF THE DRAWINGS

FIGS. 1A and 1B show that early life adversity differentiates fecal metabolite composition. FIG. 1A shows the gut metabolites cluster by PLS-DA. FIG. 1B shows the fold change of significant metabolites after FDR correction, q<0.05. Errors bars represent mean+/−SEM.

FIGS. 2A and 2B show that early life adversity differentiates brain connectivity. FIG. 2A shows brain connectivity clusters by SPLS-DA. FIG. 2B shows the significant regions after FDR correction, q<0.05. Error bars represent mean+/−SEM.

FIG. 3A shows that early life adversity interacts with clinical variables, gut metabolites and brain connectivity. After FDR correction, all variables q<0.05. Red line=positive correlation; blue line=negative correlation.

FIG. 3B shows that early life adversity impacts multiple brain networksbrain regions: SupFG/S: superior frontal gyrus and sulcus, PreCG: precentral gyms, PostCG: postcentral gyms, PaCL: paracentral lobule, pINS: posterior insula; Thal: thalamus, pACC: pregenual anterior cingulate cortex, MOcG: middle occipital gyms, CoS-LinS: medial occipito-temporal sulcus (collateral sulcus) and lingual sulcus, IPL: inferior parietal lobule, aINS: anterior insula, PrCun: precuneus, SupTGLp: lateral aspect of the superior temporal gyms, MTG: middle temporal gyms, InfTG: inferior temporal gyms, MedOrS: medial orbital sulcus (olfactory sulcus), RG: gyms rectus (straight gyms). (A color version of this figure is available at on the world wide web at .ncbi.nlm.nih.gov/pmc/articles/PMC8170500/.)

FIG. 3C shows that early life adversity interacts with clinical variables, gut metabolites and brain connectivity N=128 total, Low ETI group N=76, High ETI group N=52. p-value significant<0.05. Q-values derived from FDR correction, q-value significant<0.05. Red Line: Significant associations in the High ETI group (ETI Total>4). Green Line: Significant associations in the High ETI group (ETI Total<=4). Grey Line: Significant associations in the whole sample. Networks: SMN: sensorimotor, DMN: default mode, SAL: salience, CEN: central executive, CAN: central autonomic, ERN: emotion regulation, OCC: occipital. Brain Regions: SupFG: superior frontal gyms, SupCirinS: superior segment of the circular sulcus of the insula, PosCG: postcentral gyms, IntPS TrPS: intraparietal sulcus (interparietal sulcus) and transverse parietal sulci, InfTG: inferior temporal gyms, SupTGLp: lateral aspect of the superior temporal gyms, SupFS: superior frontal sulcus, PaCL/S: paracentral lobule and sulcus, Thal: thalamus, ATrCoS: anterior transverse collateral sulcus, RG: straight gyms (gyms rectus), ACgG S: anterior part of the cingulate gyms and sulcus, InfCirInS: inferior segment of the circular sulcus of the insula; CoS LinS: medial occipito-temporal sulcus (collateral sulcus) and lingual sulcus. Clinical Variables: ETI: early traumatic inventory; BMI: body mass index, PSS: Perceived Stress Scale, HADS: Hospital Anxiety and Depression Scale. (A color version of this figure is available at on the world wide web at .ncbi.nlm.nih.gov/pmc/articles/PMC8170500/.)

FIGS. 4A-4C show that early life adversity does not differentiate alpha and beta diversity or taxonomic relative abundances. FIG. 4A shows alpha diversity metrics, from right to left: ACE (p=0.50749), Chaol (p=0.63385), Shannon (p=0.10209). FIG. 4B shows beta diversity: Bray-Curtis-based PCoA (permanova p=0.441). FIG. 4C shows the taxonomic relative abundances: top refers to phylum level, bottom refers to genus level.

### DETAILED DESCRIPTION OF THE INVENTION

It has been determined herein that certain adult gut metabolites (e.g., glutamate, gamma-methyl ester, malate, lithocholic acid sulfate, urate, and 5-oxoproline) are associated with early life adversity (ELA). Accordingly, the present invention relates, in part, to methods for identifying patients with ELA based upon a determination and analysis of amounts of such metabolites, compared to a control level. In addition, methods for preventing, treating, and/or reducing psychological distress in patients with ELA by increasing the level and/or activity of these metabolites are also provided.

### I. Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent (such as the compositions described herein) to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo. In some embodiments, the agent is orally administered. In other embodiments, the agent is administered through anal and/or colorectal route.

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The amount of a biomarker (e.g., one or more metabolites described herein) in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, activity, and the like.

The term “assigned score” refers to the numerical value designated for each of the biomarkers after being measured in a patient sample. The assigned score correlates to the absence, presence or inferred amount of the biomarker in the sample. The assigned score can be generated manually (e.g., by visual inspection) or with the aid of instrumentation for image acquisition and analysis. In certain embodiments, the assigned score is determined by a qualitative assessment, for example, detection of a fluorescent readout on a graded scale, or quantitative assessment. In certain embodiments, an “aggregate score,” which refers to the combination of assigned scores from a plurality of measured biomarkers, is determined. For example, the aggregate score may be a summation of assigned scores. Alternatively, combination of assigned scores may involve performing mathematical operations on the assigned scores before combining them into an aggregate score. In certain embodiments, the aggregate score is also referred to herein as the “predictive score.”

The term “biomarker” refers to a measurable parameter of the present invention that has been determined to be predictive of (1) a subject with a specific condition (e.g., a history of ELA), or (2) of the effects of an agent or therapy described herein, either alone or in combination with at least one other therapies, on a target disease or disorder (e.g., psychological distress in patients with ELA). Biomarkers can include, without limitation, bacteria, amino acid metabolites, and clinical characteristics of a subject, including those shown in the Tables, the Examples, the Figures, and otherwise described herein. For example, a bacterial biomarker (such as at least one type of bacteria and/or metabolites described in Examples) may be detected and analyzed by any known methods, such as detecting and/or quantifying the bacteria and/or metabolites by in vivo or in vitro assays or detecting bacterial-originated polynucleotides, polypeptides, and/or metabolites, etc. A metabolite biomarker (e.g., adult gut metabolites associated with ELA described in Examples, such as glutamate, gamma-methyl ester, malate, lithocholic acid sulfate, urate, and may be detected and/or quantified by any known methods for chemicals (e.g., mass spectrometry, HPLC, or NMR). A clinical biomarker (e.g., body mass index (BMI), Perceived Stress Scale (PSS Sore), Hospital Anxiety and Depression Scale Anxiety (HAD Anxiety), Hospital Anxiety and Depression Scale Depression (HAD Depression), brain connectivity measures, etc.) may be measured by any suitable methods known, e.g., the methods described in the Examples.

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). For example, any body fluid may be taken to detect and/or measure at least one biomarker described herein.

The term “control” refers to any reference standard suitable to provide a comparison to the biomarkers/products in the test sample. In certain embodiments, the control comprises obtaining a “control sample” from which product or biomarker levels are detected and compared to the product or biomarker levels from the test sample. Such a control sample may comprise any suitable sample, including but not limited to a sample from a control subject (can be stored sample or previous sample measurement) with a known outcome; normal tissue or cells isolated from a subject, such as a normal subject or the subject with a low Early Traumatic Inventory-Self Report (ETI-SR) (e.g., ETI 4), cultured primary cells/tissues isolated from a subject such as a normal subject or the subject with with a low Early Traumatic Inventory-Self Report (ETI-SR) (e.g., ETI 4), a tissue or cell sample isolated from a normal subject, or a primary cells/tissues obtained from a depository. In other preferred embodiments, the control may comprise a reference standard expression product or biomarker level from any suitable source, including but not limited to housekeeping genes, an expression product level range from normal tissue (or other previously analyzed control sample), a previously determined expression product level range within a test sample from a group of patients, or a set of patients with a certain outcome or receiving a certain treatment. It will be understood by those of skill in the art that such control samples and reference standard product or biomarker levels can be used in combination as controls in the methods of the present invention. In the former case, the specific product or biomarker level of each patient can be assigned to a percentile level of expression, or expressed as either higher or lower than the mean or average of the reference standard expression level. In other embodiments, the control may also comprise a measured value for example, average level of expression of a particular gene in a population compared to the level of expression of a housekeeping gene in the same population.

The term “comprise” or variations such as “comprises” or “comprising” will be understood to imply the inclusion of a stated integer (or components) or group of integers (or components), but not the exclusion of any other integer (or components) or group of integers (or components).

The term “increased/decrased amount” or “increased/decreased level” refers to increased or decreased absolute and/or relative amount and/or value of a biomarker (e.g., one or more metabolites described herein) in a subject, as compared to the amount and/or value of the same biomarker in the same subject in a prior time and/or in a normal and/or control subject, or a normal/control level representative of such subjects in general.

A “kit” is any manufacture (e.g., a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard. One skilled in the art can envision many such controls, including, but not limited to, common molecules. Reagents in the kit may be provided in individual containers or as mixtures of two or more reagents in a single container. In addition, instructional materials which describe the use of the compositions within the kit can be included.

The “normal” level of expression and/or activity of a biomarker is the level of expression and/or activity of the biomarker in cells of a subject, e.g., a human patient, not afflicted with ELA. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples. The same determination can be made to determine overactivity or underactivity.

In some embodiments, levels of one or more biomarkers (e.g., one or more metabolites decribed herein) are measured and compared at different time points to assess the progression of a disease or to assess the efficacy of an agent for treating a disease. Therefore, in some embodiments, a “significantly higher level” or “significantly increased level” of a biomarker refers to an expression level, amount and/or activity level in a subject sample at one point in time that is greater than the standard error of the assay employed to assess the expression level, amount and/or activity level, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression level, amount or activity level of the biomarker in a subject sample at another point in time. In some embodiments, a “significantly lower level” or “significantly decreased level” of a biomarker refers to an expression level, amount and/or activity level in a subject sample at one point in time that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level, amount or activity level of the biomarker in a subject sample at another point in time.

The term “pre-determined” biomarker amount and/or activity measurement(s) may be a biomarker amount and/or activity measurement(s) used to, by way of example only, evaluate a subject that may be selected for a particular treatment, evaluate a response to a treatment such as using a composition described herein, alone or in combination with other therapy to reduce pshycological distress. A pre-determined biomarker amount and/or activity measurement(s) may be determined in populations of patients with or without a disease (e.g., a history of ALE and/or psychological distress). The pre-determined biomarker amount and/or activity measurement(s) can be a single number, equally applicable to every patient, or the pre-determined biomarker amount and/or activity measurement(s) can vary to reflect differences among specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In certain embodiments, the amounts determined and/or compared in a method described herein are based on absolute measurements. In other embodiments, the amounts determined and/or compared in a method described herein are based on relative measurements, such as ratios (e.g., serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different subject for whom a subject selection is being assessed. In some embodiments, the pre-determined biomarker amount and/or activity measurement(s) can be obtained from a previous assessment of the same subject. In such a manner, the progress of the selection of the patient can be monitored over time. In addition, the control can be obtained from an assessment of another subject or multiple subjects, e.g., selected groups of subjects. In such a manner, the extent of the selection of the subject for whom selection is being assessed can be compared to suitable other subjects, e.g., other subjects who are in a similar situation to the human of interest, such as those suffering from similar or the same condition(s) and/or of the same ethnic group.

As used herein, a therapeutic that “prevents” a condition refers to a composition that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample. For example, the compositions or methods described herein may prevent psychological distress in patients with a history of ELA.

The term “prognosis” includes a prediction of the probable course and outcome of psychological distresss in patients with a history of ALE or the likelihood of recovery from the disease.

The term “prodrug” is intended to encompass compounds which, under physiologic conditions, are converted into the therapeutically active agents of the present invention (e.g., theobromine). A common method for making a prodrug is to include one or more selected moieties which are hydrolyzed under physiologic conditions to reveal the desired molecule. In other embodiments, the prodrug is converted by an enzymatic activity of the subject. For example, esters or carbonates (e.g., esters or carbonates of alcohols or carboxylic acids) are preferred prodrugs of the present invention. In certain embodiments, some or all of the compounds of the present invention (e.g., metabolites described herein) in a formulation represented above can be replaced with the corresponding suitable prodrug, e.g., wherein a hydroxyl in the parent compound is presented as an ester or a carbonate or carboxylic acid present in the parent compound is presented as an ester. A prodrug of theobromine may be formed, for example, by replacing the imide hydrogen with a labile group, such as a methoxymethyl group or a p-methoxyphenyl group.

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of a therapeutically effective substance (e.g., metabolites described herein) of this disclosure. These salts can likewise be prepared in situ during the final isolation and purification of the respiration uncoupling agents, or by separately reacting the purified respiration uncoupling agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al. (1977) “Pharmaceutical Salts”, J. Pharm. Sci. 66:1-19).

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one biomarker in the sample.

The term “synergistic effect” refers to the combined effect of two or more agents described herein can be greater than the sum of the separate effects of any one of agents alone.

The terms “subject” refer to either a human or a non-human animal. This term includes mammals such as humans, primates, livestock animals (e.g., bovines, porcines), companion animals (e.g., canines, felines) and rodents (e.g., mice, rabbits and rats).

“Treating” a disease in a subject or “treating” a subject having a disease refers to subjecting the subject to a pharmaceutical treatment, e.g., the administration of a drug, such that at least one symptom of the disease is decreased or prevented from worsening.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

Unless otherwise defined herein, scientific and technical terms used in this application shall have the meanings that are commonly understood by those of ordinary skill in the art. Generally, nomenclature and techniques relating to chemistry, molecular biology, cell and cancer biology, immunology, microbiology, pharmacology, and protein and nucleic acid chemistry, described herein, are those well-known and commonly used in the art.

### II. Subjects

In certain embodiments, the subject suitable for the compositions and methods disclosed herein is a mammal (e.g., mouse, rat, primate, non-human mammal, domestic animal, such as a dog, cat, cow, horse, and the like), and is preferably a human. In other embodiments, the subject is an animal model of ALE.

In other embodiments of the methods of the present invention, the subject has not undergone treatment for psychological distress associated with ALE (e.g., depression, anxiety, perceived stress, etc.). In still other embodiments, the subject has undergone treatment for psychological distress associated with ALE (e.g., depression, anxiety, perceived stress, etc.).

The methods of the present invention can be used to treat psychological distress in subjects with a history of ALE such as those described herein, and/or determine the responsiveness to a composition described herein, alone or in combination with other therapies.

### III. Uses and Methods of the Present Invention

In some aspects, provided herein are a variety of diagnostic, prognostic, and therapeutic methods. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

### (1) Predictive Medicine

The present invention can pertain to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a biomarker described herein in the context of a biological sample (e.g., blood, serum, cells, stool, or tissue) to thereby determine whether an individual has a history of ALE, or whether an agent is likely to be effective for treating or reducing psychological distress in a subject with a history of ELA. Such assays can be used for prognostic or predictive purpose alone, or can be coupled with a therapeutic intervention to thereby prophylactically treat an individual prior to the onset or after recurrence of a disorder characterized by or associated with biomarker level or activity. The skilled artisan will appreciate that any method can use one or more (e.g., combinations) of biomarkers described herein, such as those in the tables, figures, examples, and otherwise described in the specification.

### (2) Diagnostic Assays

The present invention provides, in part, methods, systems, and code for accurately classifying whether a biological sample (e.g., from a subject) or a subject is associated with ALE. In some embodiments, the present invention is useful for classifying a sample (e.g., from a subject) or a subject as associated with ALE as disclosed herein using a statistical algorithm and/or empirical data (e.g., the amount or activity of a biomarker described herein, such as in the tables, figures, examples, and otherwise described in the specification).

An exemplary method for detecting the amount or activity of a biomarker described herein, and thus useful for classifying whether a sample or a subject is assocaited with ALE involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in series. In other embodiments, the amount of the biomarker (e.g., metabolites described herein) in the biological sample is measured by standard methods used to measure chemicals, including but not limited to, mass spectrometry, NMR, chromatography, and HPLC.

In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely therapy responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In other embodiments, the diagnosis of a subject is followed by administering to the individual a therapeutically effective amount of a defined treatment based upon the diagnosis.

In some embodiments, the methods further involve obtaining a control biological sample (e.g., biological sample from a subject who does not have a history of ALE, or has a ETI-SR score 4), a biological sample from the subject during remission, or a biological sample from the subject during treatment for developing psychnological distress assocaited with ALE progressing.

### (3) Prognostic Assays

The diagnostic methods described herein can furthermore be utilized to identify subjects having a history of ALE or at risk of developing psychological distress assocaited with ALE that is likely or unlikely to be responsive to a composition as disclosed herein. The assays described herein, such as the preceding diagnostic assays or the following assays, can be utilized to identify a subject having or at risk of developing a disorder associated with a misregulation of the amount or activity of at least one biomarker described herein. Alternatively, the prognostic assays can be utilized to identify a subject having or at risk for developing a disorder associated with a misregulation of the at least one biomarker described herein. Furthermore, the prognostic assays described herein can be used to determine whether a subject can be administered a composition as disclosed herein and/or an additional therapeutic regimen to treat a disease or disorder associated with the aberrant biomarker expression or activity.

An “isolated” or “purified” biomarker (e.g., bacteria or metabolic products) is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

### (4) Sampling Methods

In some embodiments, biomarker amount and/or activity measurement(s) in a sample from a subject is compared to a predetermined control (standard) sample. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment, evaluate a response to a composition as disclosed herein, alone or in combination with one or more additional therapies. A pre-determined biomarker amount and/or activity measurement(s) may be determined in populations of patients with or without a history of ALE. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In some embodiments, the amounts determined and/or compared in a method described herein are based on absolute measurements.

The term “disease” includes a disorder and/or a status of a subject when reducing psychological distress will be generally beneficial to at least the health (e.g., both physical and psychological health) of the subject. For example, depression, anxiety, stress (e.g., self-perceived stress), negative mood, and other negative emotional states is included in the scope of “diseases” described herein, whether or not it fits in the medical definition of a disease according to a medical professionnal.

In other embodiments, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of anti-obesity or weight loss therapy. Post-treatment biomarker measurement can be made at any time after initiation of therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of therapy, and even longer toward indefinitely for continued monitoring. Treatment can comprise, e.g., a therapeutic regimen comprising a composition as disclosed herein, or further in combination with other agents.

The pre-determined biomarker amount and/or activity measurement(s) can be any suitable standard. For example, the pre-determined biomarker amount and/or activity measurement(s) can be obtained from the same or a different human for whom a patient selection is being assessed. In some embodiments, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker amount and/or activity measurement(s) from the pre-determined level is about 0.1, 0.2, 0.3, 0.4, 0.5, 0.6, 0.7, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In preferred embodiments, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In some embodiments, the sample is serum, plasma, urine, or stool. In other embodiments, the sample is stool.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CLEF), capillary isotachophoresis (cITP) and capillary electrochromatography (CEC). CE techniques can be coupled to electrospray ionization through the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CLEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### (5) Treatment Methods

In some aspects, provided herein are methods of treating or reducing psychological distress in a subject with a history of ELA, comprising administering to the subject an agent that increases the level and/or activity of at least one metabolite selected from glutamate, gamma-methyl ester, 5-oxoproline, malate, lithocholic acid sulfate, and urate. Such agents may include synthesized glutamate, gamma-methyl ester, 5-oxoproline, malate, lithocholic acid sulfate, and urate, or a closely-related analogue, a prodrug, or a pharmaceutically acceptable salt of glutamate, gamma-methyl ester, 5-oxoproline, malate, lithocholic acid sulfate, or urate. Such agents may also include a probiotic bacterium, such as Lactobacillus plantarum or related large volume glutamate producers that can increase the production of the metabolites described herein. Such agents may also include microbiota enriched in Prevotella. Such agents may also be probiotic supplements (e.g., fermented foods) that contain these probiotic bacteria.

In some embodiments, the composition used in the methods described herein is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies. The composition may be a fermented food product, such as, but not limited to, a fermented milk product. Non-limiting examples of fermented food products include kombucha, sauerkraut, pickles, miso, tempeh, natto, kimchi, raw cheese, and yogurt. The composition may also be a food additive, such as, but not limited to, an acidulent (e.g., vinegar). Food additives can be divided into several groups based on their effects. Non-limiting examples of food additives include acidulents (e.g., vinegar, citric acid, tartaric acid, malic acid, fumaric acid, and lactic acid), acidity regulators, anticaking agents, antifoaming agents, foaming agents, antioxidants (e.g., vitamin C), bulking agents (e.g., starch), food coloring, fortifying agents, color retention agents, emulsifiers, flavors and flavor enhancers (e.g., monosodium glutamate), flour treatment agents, glazing agents, humectants, tracer gas, preservatives, stabilizers, sweeteners, and thickeners.

In certain embodiments, the bacteria disclosed herein are administered in conjunction with a prebiotic to the subject. Prebiotics are carbohydrates which are generally indigestible by a host animal and are selectively fermented or metabolized by bacteria. Prebiotics may be short-chain carbohydrates (e.g., oligosaccharides) and/or simple sugars (e.g., mono- and di-saccharides) and/or mucins (heavily glycosylated proteins) that alter the composition or metabolism of a microbiome in the host. The short chain carbohydrates are also referred to as oligosaccharides, and usually contain from 2 or 3 and up to 8, 9, 10, 15 or more sugar moieties. When prebiotics are introduced to a host, the prebiotics affect the bacteria within the host and do not directly affect the host. In certain aspects, a prebiotic composition can selectively stimulate the growth and/or activity of one of a limited number of bacteria in a host. Prebiotics include oligosaccharides such as fructooligosaccharides (FOS) (including inulin), galactooligosaccharides (GOS), trans-galactooligosaccharides, xylooligosaccharides (XOS), chitooligosaccharides (COS), soy oligosaccharides (e.g., stachyose and raffinose) gentiooligosaccharides, isomaltooligosaccharides, mannooligosaccharides, maltooligosaccharides and mannanoligosaccharides. Oligosaccharides are not necessarily single components, and can be mixtures containing oligosaccharides with different degrees of oligomerization, sometimes including the parent disaccharide and the monomeric sugars. Various types of oligosaccharides are found as natural components in many common foods, including fruits, vegetables, milk, and honey. Specific examples of oligosaccharides are lactulose, lactosucrose, palatinose, glycosyl sucrose, guar gum, gum Arabic, tagalose, amylose, amylopectin, pectin, xylan, and cyclodextrins. Prebiotics may also be purified or chemically or enzymatically synthesized.

### IV. Pharmaceutical Compositions

The present invention provides pharmaceutically acceptable compositions of the agents disclosed herein. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles.

In some embodiments, compositions described herein may be used for oral administration to the gastrointestinal tract, directed at the objective of introducing the probiotic bacteria to tissues of the gastrointestinal tract. The formulation for a therapeutic composition of the present invention may also include other probiotic agents or nutrients which promote spore germination and/or bacterial growth. An exemplary material is a bifidogenic oligosaccharide, which promotes the growth of beneficial probiotic bacteria. In certain embodiment, the probiotic bacterial strain is combined with a therapeutically-effective dose of an (preferably, broad spectrum) antibiotic, or an anti-fungal agent. In some embodiments, the compositions described herein are encapsulated into an enterically-coated, time-released capsule or tablet. The enteric coating allows the capsule/tablet to remain intact (i.e., undisolved) as it passes through the gastrointestinal tract, until after a certain time and/or until it reaches a certain part of the GI tract (e.g., the small intestine). The time-released component prevents the “release” of the probiotic bacterial strain in the compositions described herein for a pre-determined time period.

The therapeutic compositions of the present invention may also include known antioxidants, buffering agents, and other agents such as coloring agents, flavorings, vitamins or minerals.

In some embodiments, the therapeutic compositions of the present invention are combined with a carrier which is physiologically compatible with the gastrointestinal tissue of the species to which it is administered. Carriers can be comprised of solid-based, dry materials for formulation into tablet, capsule or powdered form; or the carrier can be comprised of liquid or gel-based materials for formulations into liquid or gel forms. The specific type of carrier, as well as the final formulation depends, in part, upon the selected route(s) of administration. The therapeutic composition of the present invention may also include a variety of carriers and/or binders. A preferred carrier is micro-crystalline cellulose (MCC) added in an amount sufficient to complete the one gram dosage total weight. Carriers can be solid-based dry materials for formulations in tablet, capsule or powdered form, and can be liquid or gel-based materials for formulations in liquid or gel forms, which forms depend, in part, upon the routes of administration. Typical carriers for dry formulations include, but are not limited to: trehalose, malto-dextrin, rice flour, microcrystalline cellulose (MCC) magnesium sterate, inositol, FOS, GOS, dextrose, sucrose, and like carriers. Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like). Preferably, water-based carriers possess a neutral pH value (i.e., pH 7.0). Other carriers or agents for administering the compositions described herein are known in the art, e.g., in U.S. Pat. No. 6,461,607.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of one or more bacterial strains as disclosed herein.

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### EXAMPLES

### Example 1

### Materials and Methods for Examples 2-8

### Subjects

The sample was comprised of 128 right-handed participants (43 males and 85 females), with the absence of significant medical or psychiatric conditions. Participants were excluded for the following: pregnant or lactating, substance use, abdominal surgery, tobacco dependence (half a pack or more daily), extreme strenuous exercise (>8 h of continuous exercise per week), current or past psychiatric illness, and major medical or neurological conditions. Subjects taking medications that interfere with the CNS or using analgesic drugs regularly were excluded. Participants were also excluded for use of antibiotics in the past 3 months. Since female sex hormones such as estrogen are known to effect brain structure and function, we used only females who were premenopausal.

All procedures complied with the principles of the Declaration of Helsinki and were approved by the Institutional Review Board (16-000187, 15-001591) at the University of California, Los Angeles's Office of Protection for Research Subjects. All participants provided written informed consent.

### Questionnaires

ELA was measured using the Early Traumatic Inventory-Self Report (ETI-SR) (40), a 27-item questionnaire. This questionnaire assesses the histories of childhood traumatic and adverse life events that occurred before the age of 18 years old and covers four domains: general trauma (11 items), physical punishment (5 items), emotional abuse (5 items), and sexual abuse (6 items); see supplementary methods for details. The ETI-SR instrument was chosen due to its psychometric properties, ease of administration, time efficiency, and ability to measure ELAs in multiple domains (41). For subsequent analyses, participants were split into two groups: “High ETI” (ETI-SR total >4) and “Low ETI” (ETI-SR total≤4).

Additional questionnaires included the Perceived Stress Scale (33) and the Hospital Anxiety and Depression (33) Scale. The PSS is a 10-item scale used to measure stressful demands in a given situation, indicating that demands exceed ability to cope (42). The questions are based on subjects reporting the frequency of their feelings within the past week to each question, which are scored on a scale of 0 (never) to 4 (very often) (42). The HAD scale is a 14-item scale used to measure anxiety and depression (43). The questions are scored on a scale of 0 to 3, corresponding to how much the individual identifies with the question for the past week. (43, 44).

Diet was assessed through self-reported questionnaires, where participants were asked to select which diet they consumed on a regular basis; see supplementary methods for details.

### Gut Microbiome

### Collection and Storage

Participants were given “at home kits” with specific instructions regarding time of stool collection (e.g. time of day and within 2-3 days before the MRI scan). In addition, 2-3 consecutive diet diaries were collected from the time of enrollment to the time of the MRI scan and stool collection (1 weekday and 1 weekend). Subjects were asked to collect the stool before the first meal of the day. If participants were on antidiarrheal or laxatives, they were asked to refrain from use for 2-3 days before the sample collection. Any deviation from the stool sample collection were documented in order to account for in the analyses. Fecal samples were stored at -80° C., then ground into a coarse powder by mortar and pestle under liquid nitrogen and aliquoted for DNA extraction and metabolomics. See supplementary methods for details on DNA extraction and 16S rRNA gene sequencing.

### Fecal Metabolomics

Fecal aliquots were shipped to Metabolon, Inc., and run as a single batch on their global HD4 metabolomics platform; see supplementary methods for details on processing and results were provided as scaled, imputed abundances of 872 known compounds (45). Missing values of raw data were filled up using median values, and ineffective peaks were removed through the interquartile range denoising method. In addition, the internal standard normalization method was employed in the data analysis. The dataset for the multiple classification analysis was compiled from the metabolite profiling results and a 3D matrix involving metabolite numbers, sample names, and normalized peak intensities were fed into the MetaboAnlyst web software 3.0 (available on the world wide web at metaboanalyst.ca) (46).

### Magnetic Resonance Imaging

Whole brain structural and functional (resting state) data was acquired using a 3.0T Siemens Prisma MRI scanner (Siemens, Erlangen, Germany). Detailed information on the standardized acquisition protocols, quality control measures, and image preprocessing are provided in previously published studies (15, 47-51). See supplementary methods for details on acquisition, preprocessing of images, structural image parcellation, and construction of functional connectivity matrices for each subject, for 430 parcellated regions.

### Statistical Analysis

### Sparse Partial Least Squares—Discriminate Analysis

A partial least squares-discriminant analysis (PLS-DA) was conducted in R (Boston, MA) to explore the group difference between high vs. low ETI groups by incorporating known classifications for the metabolites. Similarly, a sparse PLS-DA for whole brain resting state connectivity was run to understand the classification in brain signatures related to high vs. low ETI. In order to prevent overfitting of the model, we ran permutation tests as previously published (52, 53). The metabolites with values of the first component of variable importance projection (VIP) greater than 1.0 were assessed, indicating the estimate of the importance of each metabolite used in the PLS model. The brain connectivity regions/brain signatures from the two components of the weighted design matrix and contributing to the discrimination between the two groups were summarized using the top variable loadings on the individual dimensions/components and VIP coefficients. T-tests using contrasts in a general linear model controlling for age, BMI, diet, and sex were conducted. P-values were adjusted for with the Benjamini-Hochberg false discovery rate (FDR) procedure and significant q-values, were reported (54). For metabolites those with VIPs>1.0 and q<0.05 were selected as significantly different between the two groups. The fold change was also calculated to investigate the difference by comparing the mean value of the peak area obtained between the two groups.

### Tripartite Network Analysis

Tripartite network analysis was performed to integrate information from three data sets:

1) stool-derived metabolites 2) clinical data (ETI, PSS, HAD Anxiety, HAD Depression) and 3) functional connectivity brain data. The interaction between the phenome (clinical measures), microbiome (stool-derived metabolites) and connectome (brain connectivity) was determined by computing Spearman correlations between different data types in R v.3.6.2, controlling for sex. FDR correction was applied to generate q-values. Cytoscape v.3.7.2 was used to visualize and construct brain, symptom, and gut-derived metabolite interaction networks thresholded at q <0.05. We present the networks by placing nodes of the same type together and displaying connecting edges representing correlations. A red edge indicates a positive correlation, and a blue edge indicates a negative correlation.

### Supplementary Methods

### Questionnaires

The ETI-SR subscales contain general traumatic events, physical abuse, emotional abuse, and sexual abuse. General traumatic events comprise a range of stressful and traumatic events that can be mostly secondary to chance events. Sample items on this scale include death of a parent, discordant relationships or divorce between parents, or death or sickness of a sibling or friend. Physical abuse involves physical contact, constraint, or confinement, with intent to hurt or injure. Sample items on the physical abuse subscale include being spanked by hand or being hit by objects. Emotional abuse is verbal communication with the intention of humiliating or degrading the victim. Sample items on the ETI-SR emotion subscale include the following, “Often put down or ridiculed,” or “Often told that one is no good.” Sexual abuse is unwanted sexual contact performed solely for the gratification of the perpetrator or for the purposes of dominating or degrading the victim. Sample items on the sexual abuse scale include being forced to pose for suggestive photographs, to perform sexual acts for money, or coercive anal sexual acts against one's will.

The self-reported diet questionnaire included the following options: Standard American (characterized by high consumption of processed, frozen, and packaged foods, pasta and breads, and red meat; vegetables and fruits are not consumed in large quantities), Modified American (high consumption of whole grains including some processed, frozen, and packaged foods; red meat is consumed in limited quantities; vegetables and fruit are consumed in moderate to large quantities), Mediterranean (high consumption of fruits, vegetables, beans, nuts, and seeds; olive oil is the key monounsaturated fat source; dairy products, fish, and poultry are consumed in low to moderate amounts and little red meat is eaten), and all other diets that do not fit into the above categories.

16s rRNA Gene Sequencing

DNA extraction with bead beating was performed using the QIAGEN Powersoil kit. The V4 hypervariable region of the 16S rRNA gene was then amplified using the 515F and 806R primers to generate a sequencing library according to a published protocol (98). The library underwent 2x250 sequencing on an Illumina HiSeq 2500 to a mean depth of 250,000 merged sequences per sample. QIIME 1.9.1 was used to perform quality filtering, merge paired end reads, and cluster sequences into 97% operational taxonomic units (OTUs) (99). OTUs were classified taxonomically using the Greengenes May 2013 database at the level of domain, phylum, family, genus, and species, depending on the depth of reliable classifier assignments.

Microbial alpha diversity was assessed on datasets rarefied to equal sequencing depth (34,222) using the Chaol index of richness, Faith's phylogenetic diversity, and the Shannon index of evenness. Microbial composition was compared across samples by weighted UniFrac distances and visualized with principal coordinates analysis (100). The significance of differences in microbial composition between individuals with high or low ETI scores, adjusting for age, BMI, diet, and sex was assessed using PERMANOVA with 100,000 permutations (101). Differential abundance of microbial genera was determined using multivariate negative binomial mixed models implemented in DESeq2 that included age, BMI, diet, and sex as covariates (102). P-values were adjusted for multiple hypothesis testing to generate q-values, with a significance threshold of q<0.05.

### Fecal Metabolomics

Analyses at Metabolon, Inc. involved running methanol extracted samples through ultrahigh performance liquid chromatography-tandem mass spectroscopy under four separate chromatography and electrospray ionization conditions to separate compounds with a wide range of chemical properties. Compounds were identified by comparison of spectral features to Metabolon's proprietary library that includes MS/MS spectral data on more than 3300 purified standards. Study specific technical replicates generated by pooling aliquots of all samples were used to measure total process variability (median relative standard deviation 13%). Results were provided as scaled, imputed abundances of 872 known compounds.

### MRI Acquisition and Preprocessing

### Structural MRI Acquisition

High resolution T1-weighted images were acquired: echo time/ repetition time (TE/TR)=3.26 ms/2200 ms, field of view (103)=220×220 mm slice thickness=1 mm, 176 slices, 256×256 voxel matrices, and voxel size=0.86×0.86×1 mm.

### Functional MRI Acquisition

Resting-state scans were acquired with eyes closed and an echo planar sequence with the following parameters: TE/TR=28ms/2000ms, flip angle=77 degrees, scan duration=8 m6 s−10 m6 s, FOV=220 mm, slices=40 and slice thickness=4.0 mm, and slices were obtained with whole-brain coverage.

### Preprocessing of MRI Images

Preprocessing and quality control of functional images was done using SPM-12 software (Welcome Department of Cognitive Neurology, London, UK). The first two volumes were discarded to allow for stabilization of the magnetic field. Slice timing correction was performed first, followed by rigid six-degree motion-correction for the six realignment parameters. The motion correction parameters in each degree were examined for excessive motion. If any motion was detected above 2 mm translation or 2° rotation, the scan, along with the paired structural scan was discarded. In order to robustly take account the effects of motion, root mean squared (33) realignment estimates were calculated as robust measures of motion using publicly available MATLAB code from GitHub (28). Any subjects with a greater RMS value than 0.25 was not included in the analysis (28). The resting state images were then co-registered to their respective anatomical T1 images. Each T1 image was then segmented and normalized to a smoothed template brain in Montreal Neurological Institute (33) template space. Each subject's T1 normalization parameters were then applied to that subject's resting state image, resulting in an MNI space normalized resting state image. The resulting images were smoothed with 5 mm³ Gaussian kernel. For each subject, a sample of the volumes was inspected for any artifacts and anomalies. Levels of signal dropout were also visually inspected for excessive dropout in a priori regions of interest.

### Structural Image Parcellation

T1-image segmentation and cortical and subcortical regional parcellation were conducted using Schaefer 400 atlas (104), Harvard-Oxford subcortical atlas (105-107), and the Ascending Arousal Network atlas (108). This parcellation results in the labeling of 430 regions, 400 cortical structures, 14 bilateral subcortical structures, bilateral cerebellum, and 14 brainstem nuclei (109).

### Functional Brain Connectivity Matrix Construction

To summarize, all pre-processed, normalized images were entered into the CONN-fMRI functional connectivity toolbox version 17 in MATLAB (110). All images were first corrected for noise using the automatic component-based noise correction (aCompCor) method to remove physiological noise without regressing out the global signal (111) Confounds for the six motion parameters along with their first-order temporal derivatives, along with confounds emerging from white matter and cerebral spinal fluid, and first-order temporal derivatives of motion, and root mean squared (33) values of the detrended realignment estimates (33) were removed using regression. Although the influence of head motion cannot be completely removed, CompCor has been shown to be particularly effective for dealing with residual motion relative to other methods (112). The images were then band-pass filtered between 0.008 and 0.009 Hz to minimize the effects of low frequency drift and high frequency noise after CompCor regression. Connectivity matrices for each subject, consisting of all the parcellated regions in the Schaefer (104), Harvard- Oxford Subcortical (113) (107, 114, 115) and Ascending Arousal Network (33) (108) atlases, were then computed. This represents the association between two average temporal BOLD time series across all the voxels in each region. The final outputs for each subject consisted of a connectivity matrix between the 430 parcellated regions and was indexed by Fisher transformed Z correlation coefficients between each region of interest.

### Example 2

### Synopsis of the Study

Alterations in the brain-gut-microbiome (BGM) axis have been implicated in a variety of conditions and disease-states, but little is known about how it mediates the impact of early-life adversity (ELA) on development and adult health. We hypothesize that ELA acts to disrupt components of the BGM axis, such as brain functional connectivity and gut-regulated metabolites, thereby increasing susceptibility to disordered mood.

In a sample of 128 healthy subjects, ELA and current stress, depression, and anxiety were assessed using validated questionnaires. Relative fecal microbial abundance and metabolites were derived from 16S rRNA sequencing and non-targeted metabolomics. Functional brain connectivity was measured by magnetic resonance imaging. Sparse partial least squares-discriminate analysis and tripartite network analysis were used, controlling for sex, body mass index, age, and diet. Significant q-values corrected for multiple comparisons are reported.

A history of ELA was significantly associated with four gut-regulated metabolites related to glutamate pathways (5-oxoproline, malate, urate, and glutamate, gamma methyl ester), functional connectivity including regions within primarily sensorimotor, salience, and central executive networks. Significant relationships were also found directly between the four metabolites and brain connectivity measures, and with perceived stress, anxiety, and depression.

This study reveals a novel association between a history of ELA, alterations in the BGM axis, and adult negative mood and increased vulnerability to stress. We present previously unreported gut-regulated metabolite candidates that may mediate formative neural development in response to critical period stress through proposed mechanisms such as glutamatergic excitotoxicity and oxidative stress.

### Example 3

### Subject Demographics and Clinical Variables

Individuals with a history of high ELA exposure as indexed by the ETI scale had higher BMI (p<0.001) and anxiety (p=0.032) levels (Table 1). Although the high ELA group was older (p=0,2435), and reported higher levels of depression (p=0.2845), and PSS scores (p=0.069), these differences were not significant.

TABLE 1
Clinical Characteristics Associated with Early Life Adversity

| Psychological | Low ETI | | | High ETI | | | | |
|---|---|---|---|---|---|---|---|---|
| Measures | Mean | SD | N | Mean | SD | N | t | p |
| Sex | 48 Female | 28 Male | 76 | 37 Female | 15 Male | 52 | — | — |
| Age | 27.27632 | 7.551775 | 76 | 28.80769 | 7.051737 | 52 | −1.1722 | 0.2435 |
| BMI (kg/m2) | 27.42072 | 5.161639 | 76 | 30.75555 | 5.300417 | 52 | −3.5333 | 0.0006058 |
| PSS_Score | 11.17763 | 6.387333 | 76 | 13.36538 | 6.77084 | 52 | −1.8369 | 0.06904 |
| HAD_Anxiety | 3.973684 | 3.33056 | 76 | 5.326923 | 3.551974 | 52 | −2.1709 | 0.03219 |
| HAD_ | 1.815789 | 2.004731 | 76 | 2.288462 | 2.695941 | 52 | −1.0769 | 0.2845 |
| Depression | | | | | | | | |

Means and standard deviations are reported for normally distributed data. ETI threshold = 4.

### Example 4

### Early Life Adversity and Gut Microbiome Composition

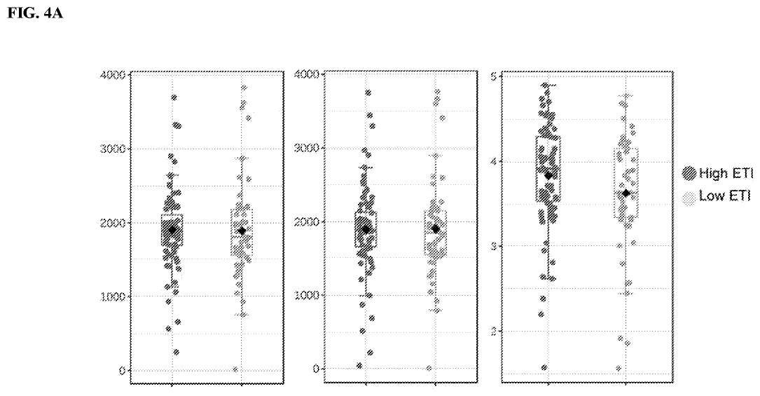

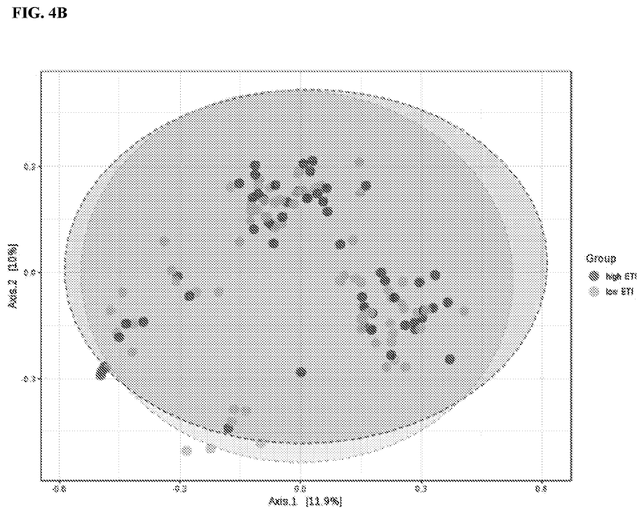

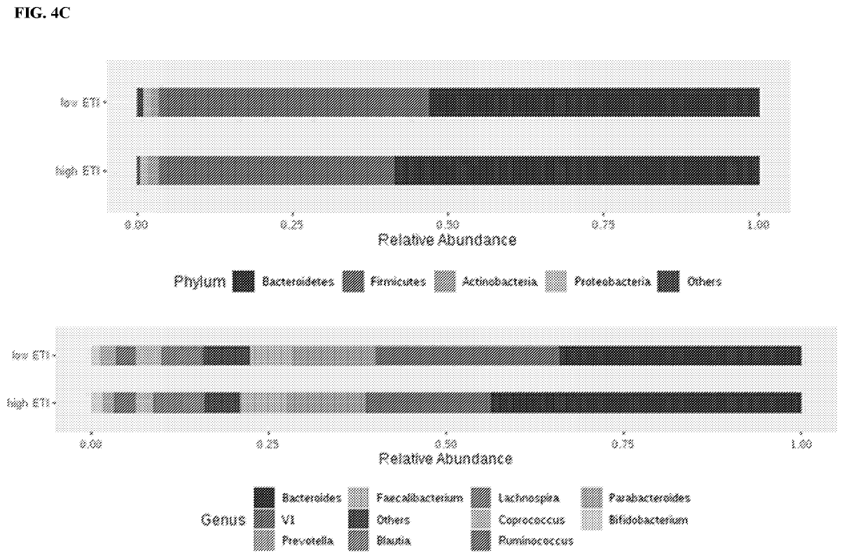

There were no significant relationships between a history of ELA exposure and microbial alpha diversity (ACE, p=0.50749; Chaol, p=0.63385; Shannon, p=0.10209) (FIG. 4A), microbial beta diversity (measured by Bray-Curtis-based PCoA; permanova p=0.441) (FIG. 4B) or relative taxonomic abundance, at either the phylum or genus levels (FIG. 4C).

### Example 5

### Early Life Adversity Associates with Adult Gut Metabolites

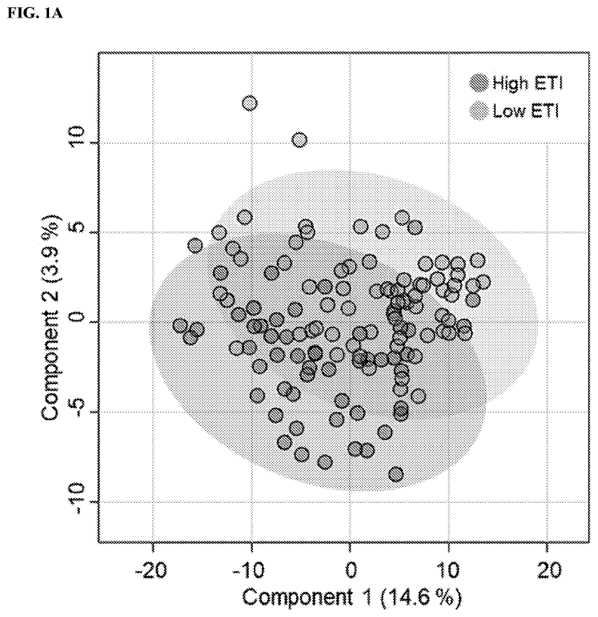

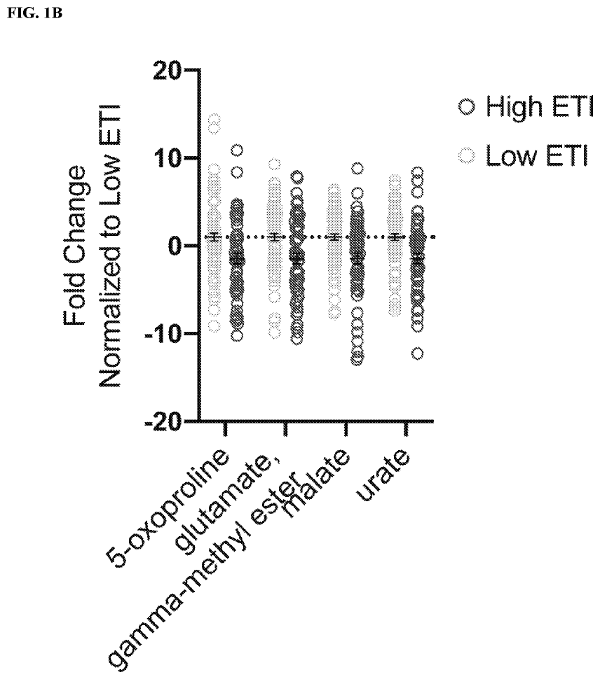

The PLS-DA of the gut metabolites showed a defined clustering, based on low or high ETI exposure (FIG. 1A). Out of 557 gut metabolites screened, 207 loaded on component 1>1.0, and were classified as “VIP” metabolites. Of this narrowed-down list of 207, 33 metabolites showed a significant relationship to ETI exposure (p<0.05), belonging to amino acid, carbohydrate, cofactors and vitamins, energy, lipid, nucleotide, and xenobiotics super pathways (Table 2). After correcting for multiple comparisons, four metabolites remained significantly correlated with ETI exposure: glutamate, gamma-methyl ester (q=0.0219), in the glutamate metabolism sub-pathway; 5-oxoproline (q=0.0200), in the glutathione metabolism sub-pathway; malate (q=0.0187), in the tricarboxylic acid (TCA) cycle sub-pathway; and urate (q=0.0358), in the purine metabolism sub-pathway. Of these four significant metabolites, all were reduced by approximately two-fold in individuals with high ETI exposure, compared to those with low ETI exposure (FIG. 1B).

TABLE 2
Gut Metabolites Associated with Early Life Adversity.

| | Super | | | | | | |
|---|---|---|---|---|---|---|---|
| VIP metabolites | Pathway | Sub Pathway | beta | se | t | p | q |
| glutamate, gamma-methyl | Amino Acid | Glutamate Metabolism | −0.4073871 | 0.17545389 | −2.3219038 | 0.02188255 | 0.0437651 |
| ester | | | | | | | |
| 5-oxoproline | Amino Acid | Glutathione Metabolism | −0.4248878 | 0.18033351 | −2.3561225 | 0.02004765 | 0.0437651 |
| formiminoglutamate | Amino Acid | Histidine Metabolism | −0.4235767 | 0.1835118 | −2.3081716 | 0.02265953 | 0.21974549 |
| N6-formyllysine | Amino Acid | Lysine Metabolism | 0.42271529 | 0.18756202 | 2.25373602 | 0.02598334 | 0.21974549 |
| N,N,N-trimethyl-5- | Amino Acid | Lysine Metabolism | −0.46249 | 0.18492285 | −2.500989 | 0.01369958 | 0.21974549 |
| aminovalerate | | | | | | | |
| N,N-dimethyl-5- | Amino Acid | Lysine Metabolism | −0.5191136 | 0.18394485 | −2.8221156 | 0.0055652 | 0.21974549 |
| aminovalerate | | | | | | | |
| N6,N6-dimethyllysine | Amino Acid | Lysine Metabolism | 0.41290892 | 0.18856914 | 2.18969514 | 0.03043327 | 0.21974549 |
| N1,N12-diacetylspermine | Amino Acid | Polyamine Metabolism | 0.41852831 | 0.18369697 | 2.27836266 | 0.02443015 | 0.21974549 |
| (N(1) + N(8))- | Amino Acid | Polyamine Metabolism | 0.40174917 | 0.18454484 | 2.17697312 | 0.03139221 | 0.21974549 |
| acetylspermidine | | | | | | | |
| diacetylspermidine* | Amino Acid | Polyamine Metabolism | 0.46783857 | 0.18617943 | 2.51283712 | 0.01327011 | 0.21974549 |
| lactate | Carbohydrate | Glycolysis, | −0.3895903 | 0.18530799 | −2.1023933 | 0.03755801 | 0.40100205 |
| | | Gluconeogenesis, and | | | | | |
| | | Pyruvate Metabolism | | | | | |
| xanthopterin | Cofactors and | Pterin Metabolism | −0.4097143 | 0.18587729 | −2.2042192 | 0.02936973 | 0.25024459 |
| | Vitamins | | | | | | |
| malate | Energy | TCA Cycle | −0.550106 | 0.18242433 | −3.0155299 | 0.00311714 | 0.01870284 |
| tricarballylate | Energy | TCA Cycle | −0.3784318 | 0.18770761 | 2.0160706 | 0.04597119 | 0.13791356 |
| N-behenoy1- | Lipid | Ceramides | −0.420415 | 0.18833109 | 2.2323186 | 0.0274037 | 0.28225958 |
| sphingadienine | | | | | | | |
| (d18:2/22:0)* | | | | | | | |
| LAHSA (18:2/OH-18:0)* | Lipid | Fatty Acid Hydroxyl | −0.404596 | 0.18411446 | −2.1975245 | 0.02985586 | 0.28225958 |
| | | Fatty Acid | | | | | |
| dihydroorotate | Lipid | Fatty Acid | −0.4849598 | 0.1866478 | −2.5982616 | 0.010514 | 0.2348126 |
| | | Metabolism(Acyl | | | | | |
| | | Carnitine) | | | | | |
| azelate (nonanedioate; | Lipid | Fatty Acid, | −0.3962817 | 0.18849183 | −2.1023813 | 0.03755909 | 0.28225958 |
| C9) | | Dicarboxylate | | | | | |
| maleate | Lipid | Fatty Acid, | −0.4997347 | 0.18707298 | −2.6713356 | 0.00857775 | 0.2348126 |
| | | Dicarboxylate | | | | | |
| mevalonate | Lipid | Mevalonate Metabolism | 0.40603584 | 0.18261353 | 2.22347071 | 0.02800998 | 0.28225958 |
| 1-palmitoylglycerol | Lipid | Monoacylglycerol | 0.40461289 | 0.18512536 | 2.18561563 | 0.03073796 | 0.28225958 |
| (16:0) | | | | | | | |
| pregnen-diol disulfate* | Lipid | Pregnenolone Steroids | 0.5417647 | 0.18671293 | 2.90159171 | 0.00440036 | 0.2348126 |
| lithocholic acid sulfate | Lipid | Secondary Bile Acid | 0.36709306 | 0.18199207 | 2.01708275 | 0.04586392 | 0.28225958 |
| (2) | | Metabolism | | | | | |
| sphinganine | Lipid | Sphingolipid Synthesis | −0.3844394 | 0.18824505 | −2.0422283 | 0.04326667 | 0.28225958 |
| allantoin | Nucleotide | Purine Metabolism, | −0.4235933 | 0.18497058 | −2.2900577 | 0.02372151 | 0.14837939 |
| | | (Hypo)Xanthine/Inosine | | | | | |
| | | containing | | | | | |
| urate | Nucleotide | Purine Metabolism, | −0.5687489 | 0.1821544 | 3.1223451 | 0.002237 | 0.03579198 |
| | | (Hypo)Xanthine/Inosine | | | | | |
| | | containing | | | | | |
| pseudouridine | Nucleotide | Pyrimidine Metabolism, | −0.3755576 | 0.18514554 | 2.0284453 | 0.04467428 | 0.14837939 |
| | | Uracil containing | | | | | |
| 3-(3- | Xenobiotics | Benzoate Metabolism | 0.46663811 | 0.18227361 | 2.56009686 | 0.01167445 | 0.10698157 |
| hydroxyphenyl) | | | | | | | |
| propionate | | | | | | | |
| 3-(4- | Xenobiotics | Benzoate Metabolism | 0.38913291 | 0.18784743 | 2.07153703 | 0.04039915 | 0.14139701 |
| hydroxyphenyl) | | | | | | | |
| propionate | | | | | | | |
| 3,4-dihydroxybenzoate | Xenobiotics | Benzoate Metabolism | −0.3951677 | 0.17863364 | 2.2121683 | 0.02880147 | 0.12096615 |
| piperidine | Xenobiotics | Food Component/Plant | −0.4597878 | 0.18690614 | −2.4599929 | 0.01528308 | 0.10698157 |
| sitostanol | Xenobiotics | Food Component/Plant | −0.4890977 | 0.18596025 | 2.6301194 | 0.00962601 | 0.10698157 |
| sucralose | Xenobiotics | Food Component/Plant | −0.4158159 | 0.1874585 | 2.2181756 | 0.02837839 | 0.12096615 |

Q-values derived from FDR correction.

### Example 6

### Early Life Adversity Associates with Brain Functional Connectivity

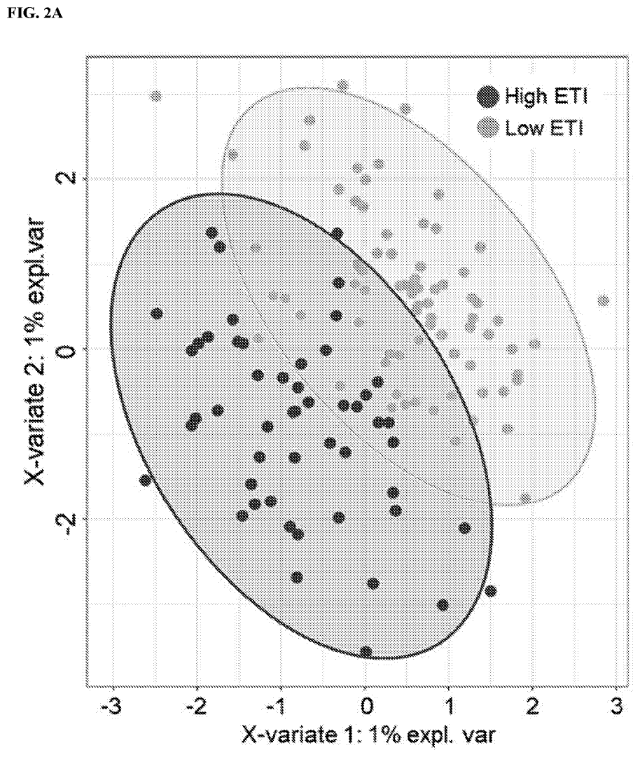

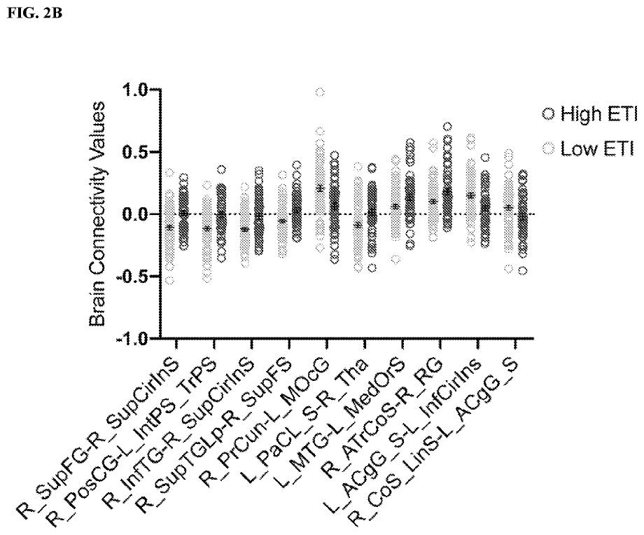

A sPLS-DA of brain functional connectivity displayed significant clustering based on low or high ETI exposure (FIG. 2A). Connectivity between eleven pairs of brain regions were significantly associated with ETI exposure (p<0.05), and after correcting for multiple comparisons, ten pairs of regions remained significant (q<0.05) (Table 3).

High ETI exposure predicted both increased and decreased connectivity between different brain networks. Increased ETI related connectivity was observed between salience, sensorimotor, central executive, default mode and central autonomic networks including: salience (superior segment of the circular sulcus of the insula) with both sensorimotor (superior frontal gyrus (q=0,0003)) and default mode (inferior temporal gyms (q=0.0003)); sensorimotor (post-central gyrus) with central executive (intraparietal sulcus, interparietal sulcus, and transverse parietal sulci (q=0.0003)); default mode (lateral aspect of the superior temporal gyrus) with sensorimotor (superior frontal sulcus (q=0.0003)); sensorimotor (paracentral lobule and sulcus) with sensorimotor (thalamus (q=0.0020)); default mode (middle temporal gyms and anterior transverse collateral sulcus) with central autonomic (medial orbital sulcus (q=0.019) and straight gyms (gyrus rectus) (q=0.0142), respectively) (FIG. 2B).

Decreased ETI related connectivity was observed between occipital, default mode, and emotion regulation networks/regions, including: default mode (precuneus) with occipital (middle occipital gyms (q=0.0003)); emotion regulation (anterior part of the cingulate gyrus and sulcus) with sensorimotor (inferior segment of the circular sulcus of the insula (q=0.0010)) and occipital (medial occipito-temporal sulcus (collateral sulcus) (q=0.0293)). Additionally, high ETI exposure predicted decreased connectivity approaching significance between default mode (precuneus) and sen.sorhnotor (precentral gyrus (q=0.0536)) (FIG. 2B).

TABLE 3
Brain Connectivity Associated with Early Life Adversity.

| | | | | | | |
|---|---|---|---|---|---|---|
| | | Network | | LOADINGS | LOADINGS | VIP |
| Network A | Region A | B | Region B | Comp 1 | Comp 2 | Comp 1 |

Brain Signature 1

| | | | | | |
|---|---|---|---|---|---|
| SMN | R_SupFG- | SAL | R_SupCirInS- | −0.77770135 | 236.1959797 |
| | Frontal | | Insular | | |
| SMN | R_PosCG- | CEN | L_IntPS_TrP | −0.46718451 | 141.8887899 |
| | Parietal | | S-Parietal | | |
| DMN | R_InfTG- | SAL | R_SupCirInS- | −0.39469134 | 119.87186 |
| | Temporal | | Insular | | |
| DMN | R_SupTG | SMN | R_SupFS- | −0.14501122 | 44.04141378 |
| | Lp- | | Frontal | | |
| | Temporal | | | | |
| DMN | R_PrCun- | OCC | L_MOcG- | 0.01047523 | 3.181436605 |
| | Parietal | | Occipital | | |

Brain Signature 2

| | | | | | |
|---|---|---|---|---|---|
| SMN | L_PaCL_ | SMN | R_Tha- | −0.66015854 | |
| | S-Parietal | | SubCortical | | |
| DMN | L_MTG- | CAN | L_MedOrS- | −0.43429941 | |
| | Temporal | | Frontal | | |
| DMN | R_ATrCoS- | CAN | R_RG- | −0.16799264 | |
| | Temporal | | Frontal | | |
| DMN | R_PrCun- | SAL | R_SupCirInS- | 0.19977185 | |
| | Parietal | | Insular | | |
| DMN | R_PrCun- | SMN | L_PRCG- | 0.25146402 | |
| | Parietal | | Frontal | | |
| DMN | R_PrCun- | SMN | R_PosCG- | 0.16181811 | |
| | Parietal | | Parietal | | |
| DMN | R_PrCun- | SMN | L_PRCG- | 0.09618968 | |
| | Parietal | | Frontal | | |
| DMN | R_PrCun- | OCC | L_MOcG- | 0.35785272 | 3.181436605 |
| | Parietal | | Occipital | | |
| ERN | L_ACgG_ | SMN | L_InfCirIns- | 0.27728102 | |
| | S-Limbic | | Insular | | |
| OCC | R_CoS_ | ERN | L_ACgG_S- | 0.06188183 | |
| | LinS- | | Limbic | | |
| | Occipital | | | | |

| | | | | | |
|---|---|---|---|---|---|
| Network A | VIP Comp 2 | t | p | q | Interpretation |

Brain Signature 1

| | | | | | |
|---|---|---|---|---|---|
| SMN | 164.5445281 | 4.2708 | 3.80E−05 | 2.99E−04 | high ETI ↑ |
| SMN | 98.84598379 | −4.1487 | 6.10E−05 | 2.99E−04 | high ETI ↑ |
| DMN | 83.50802018 | −4.1204 | 6.80E−05 | 2.99E−04 | high ETI ↑ |
| DMN | 30.68118965 | −4.0234 | 9.83E−05 | 2.99E−04 | high ETI ↑ |
| DMN | 78.00293995 | 3.9714 | 1.19E−04 | 0.0002985 | low ETI ↑ |

Brain Signature 2

| | | | | | |
|---|---|---|---|---|---|
| SMN | 143.8399457 | −3.3528 | 0.001057 | 0.00198188 | high ETI ↑ |
| DMN | 94.62818315 | −2.525 | 0.01281 | 0.019215 | high ETI ↑ |
| DMN | 36.60340855 | 2.6739 | 0.008492 | 0.01415333 | high ETI ↑ |
| DMN | 43.52768409 | 1.5621 | 0.1208 | 0.1208 | low ETI ↑ |
| DMN | 54.79073449 | 2.046 | 0.04284 | 0.05355 | low ETI ↑ |
| DMN | 35.25805801 | 1.6495 | 0.1015 | 0.11711539 | low ETI ↑ |
| DMN | 20.95847923 | 1.5666 | 0.1197 | 0.1208 | low ETI ↑ |
| DMN | 78.00293995 | 3.9714 | 0.0001194 | 0.0002985 | low ETI ↑ |
| ERN | 60.41592093 | 3.5829 | 0.000484 | 0.00103714 | low ETI ↑ |
| OCC | 13.48324456 | 2.3291 | 0.02145 | 0.02925 | low ETI ↑ |

Q-values derived from FDR correction.
SMN = sensorimotor,
DMN = default mode,
SAL = salience,
CEN = central executive,
CAN = central autonomic,
ERN = emotion regulation;
OCC = occipital.

### Example 7

### Early Life Adversity Correlates with Alterations in Brain-Gut-Microbiome Axis and Current Psychiatric Symptoms

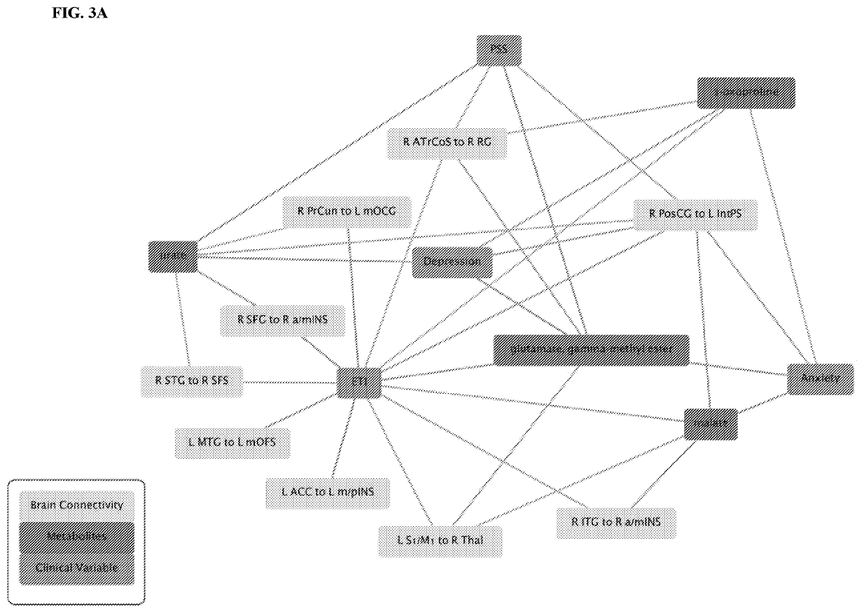

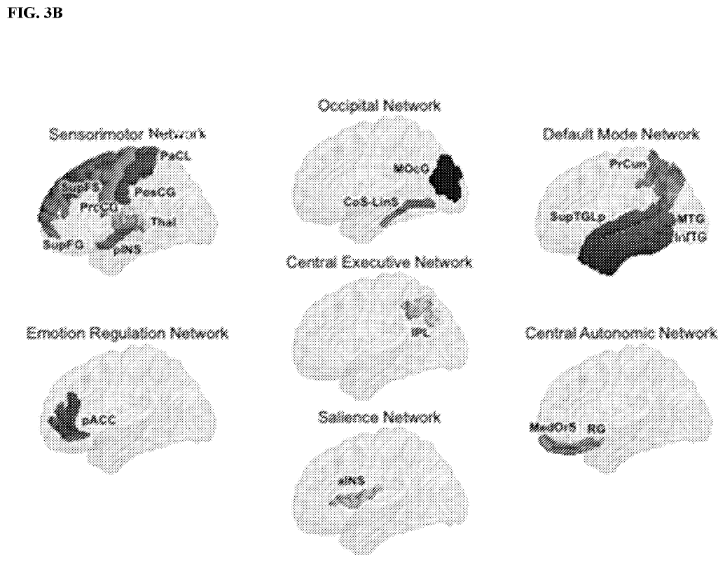

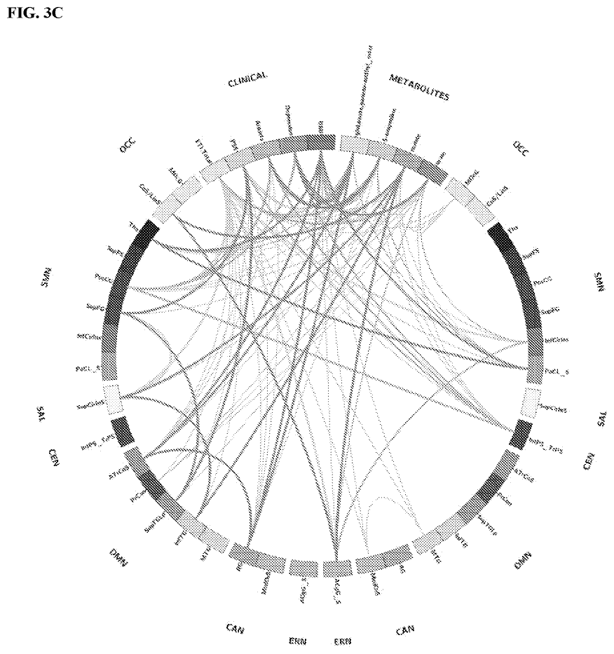

Significant relationships surviving FDR correction q<0.05) were identified between nine pairs of connected brain regions (listed in prior section), four metabolites (glutamate, gamma-methyl ester, 5-oxoproline, malate, and urate), and four clinical variables (ETI score, PSS score, HAD anxiety, and HAD depression) (Table 4; FIG. 3A, FIG. 38, FIG. 3C). ELA had positive associations with key salience (superior segment of the circular sulcus of the insula), sensorimotor (thalamus, superior frontal gyrus), and central autonomic (medial orbital sulcus) regions, but negative associations with key emotion regulation regions (anterior part of the cingulate gyrus and sulcus). In particular, connectivity between sensorimotor (post-central gyms) with central executive (intraparietal sulcus, interparietal sulcus, and transverse parietal sulci) correlated positively with all clinical measures. All four metabolites were correlated with at least one brain connectivity measure, which included mostly negative correlations with regions primarily of the sensorimotor and default mode networks, with malate and urate having the most associations. Glutamate, gamma-methyl ester was associated with all four clinical measures, whereas the other metabolites correlated significantly with only a subset.

TABLE 4
Early Life Adversity Interacts with Gut Metabolites and Brain Connectivity.

| | | | | | |
|---|---|---|---|---|---|
| | | | correlation | | |
| A | AA | B | coefficient | p | q |

Brain × Metabolites

| | | | | | |
|---|---|---|---|---|---|
| R_SupFG-Frontal-R_SupCirInS-Insular | SMN-SAL | urate | −0.2123453 | 0.01654124 | 0.02343342 |
| R_PosCG-Parietal-L_IntPS_TrPS-Parietal | SMN-CEN | malate | −0.2356181 | 0.00766044 | 0.01806054 |
| R_PosCG-Parietal-L_IntPS_TrPS-Parietal | SMN-CEN | urate | −0.2024907 | 0.02242468 | 0.02768281 |
| R_InfTG-Temporal-R_SupCirInS-Insular | DMN-SAL | malate | −0.2370112 | 0.00729842 | 0.01806054 |
| R_SupTGLp-Temporal-R_SupFS-Frontal | DMN-SMN | urate | −0.180893 | 0.04182803 | 0.04444228 |
| R_PrCun-Parietal-L_MOcG-Occipital | DMN-OCC | urate | 0.17602946 | 0.04774584 | 0.04774584 |
| L_PaCL_S-Parietal-R_Tha-SubCortical | SMN-SMN | glutamate, gamma- | −0.2190846 | 0.01333526 | 0.02159043 |
| | | methyl ester | | | |
| L_PaCL_S-Parietal-R_Tha-SubCortical | SMN-SMN | malate | −0.1922735 | 0.03033892 | 0.03438411 |
| R_ATrCoS-Temporal-R_RG-Frontal | DMN-CAN | glutamate, gamma- | −0.2260902 | 0.01059137 | 0.02000592 |
| | | methyl ester | | | |
| R_ATrCoS-Temporal-R_RG-Frontal | DMN-CAN | 5-oxoproline | −0.2077428 | 0.01909745 | 0.0251176 |

Brain × Clinical Variables

| | | | | | |
|---|---|---|---|---|---|
| R_SupFG-Frontal-R_SupCirInS-Insular | SMN-SAL | ETI_Score | 0.30200796 | 0.00055904 | 0.01806054 |
| R_PosCG-Parietal-L_IntPS_TrPS-Parietal | SMN-CEN | ETI_Score | 0.25150793 | 0.00434007 | 0.01806054 |
| R_PosCG-Parietal-L_IntPS_TrPS-Parietal | SMN-CEN | PSS_Score | 0.22387637 | 0.01139927 | 0.0203987 |
| R_PosCG-Parietal-L_IntPS_TrPS-Parietal | SMN-CEN | HAD_Anxiety | 0.23260214 | 0.00849908 | 0.01806054 |
| R_PosCG-Parietal-L_IntPS_TrPS-Parietal | SMN-CEN | HAD_Depression | 0.25446544 | 0.00388925 | 0.01806054 |
| R_InfTG-Temporal-R_SupCirInS-Insular | DMN-SAL | ETI_Score | 0.22889154 | 0.00964142 | 0.01928285 |
| R_SupTGLp-Temporal-R_SupFS-Frontal | DMN-SMN | ETI_Score | 0.22114961 | 0.01246829 | 0.02119609 |
| R_PrCun-Parietal-L_MOcG-Occipital | DMN-OCC | ETI_Score | −0.2580195 | 0.00340338 | 0.01806054 |
| L_PaCL_S-Parietal-R_Tha-SubCortical | SMN-SMN | ETI_Score | 0.20755675 | 0.01920757 | 0.0251176 |
| L_MTG-Temporal-L_MedOrS-Frontal | DMN-CAN | ETI_Score | 0.25616523 | 0.0036496 | 0.01806054 |
| L_ACgG_S-Limbic-L_InfCirIns-Insular | ERN-SMN | ETI_Score | −0.2575264 | 0.00346735 | 0.01806054 |
| R_ATrCoS-Temporal-R_RG-Frontal | DMN-CAN | ETI_Score | 0.19673609 | 0.02663029 | 0.03122171 |
| R_ATrCoS-Temporal-R_RG-Frontal | DMN-CAN | PSS_Score | 0.20194461 | 0.02279761 | 0.02768281 |

Metabolites × Clinical Variables

| | | | | |
|---|---|---|---|---|
| ETI_Score | glutamate, gamma- | −0.2403701 | 0.00648726 | 0.01806054 |
| | methyl ester | | | |
| ETI_Score | 5-oxoproline | −0.179386 | 0.04359231 | 0.04491329 |
| ETI_Score | malate | −0.2170745 | 0.01422925 | 0.02199066 |
| PSS_Score | glutamate, gamma- | −0.2594139 | 0.00322816 | 0.01806054 |
| | methyl ester | | | |
| PSS_Score | urate | −0.2338188 | 0.00815148 | 0.01806054 |
| HAD_Anxiety | glutamate, gamma- | −0.2426215 | 0.0059894 | 0.01806054 |
| | methyl ester | | | |
| HAD_Anxiety | 5-oxoproline | −0.1911046 | 0.03137984 | 0.0344166 |
| HAD_Anxiety | malate | −0.2398929 | 0.00659739 | 0.01806054 |
| HAD_Depression | glutamate, gamma- | −0.2679428 | 0.00232219 | 0.01806054 |
| | methyl ester | | | |
| HAD_Depression | 5-oxoproline | −0.2140437 | 0.01567594 | 0.02317313 |
| HAD_Depression | urate | −0.2459601 | 0.00531365 | 0.01806054 |

### Example 8

### Discussion

The current study aimed to test the hypothesis that a history of ELA is associated with altered BGM interactions impacting perceived stress, depression, and anxiety in adulthood. Our findings suggest a role for the BGM axis in influencing brain circuitry, host physiology, and susceptibility to psychological conditions in response to adversity during early life.

Early Life Adversity is Associated with Adult Gut Metabolites

We identified four fecal metabolites—urate, malate, glutamate, gamma-methyl ester, and 5-oxoproline—as significantly negatively correlated with ELA. Although a comprehensive understanding of the role of these metabolites in humans is limited, previous preclinical investigations demonstrate that some of them display sensitivity to environmental disruptors and disease (55), as well as to microbiome alterations (56). Critically, these four metabolites have been shown to be at least partially microbiota-derived (56-59). In particular, high levels of urate in human plasma have been related to protection against Parkinson's Disease (60), as well as associated with an enterotype dominated by Prevotella (57); while 5-oxoproline has been shown to be increased in the liver of mice given fecal-microbial transplants from patients with major depressive disorder (59), while levels were decreased in the serum of rats treated with antibiotics (58).

One potential mechanism posits an important role for these metabolites in mediating the relationship between ELA and pathways important in oxidative stress. ELA has been previously linked to oxidative stress and cellular aging (61). In a sample of healthy women, oxidative stress index was positively associated with perceived stress and telomere length (62). Similarly, a history of childhood maltreatment successfully predicts shorter telomeres (63, 64) and greater mitochondrial DNA copies (63), a marker of oxidative damage, in healthy adults. Notably, these four metabolites of interest have previously been implicated in and described within the context of oxidative stress in animal models (65-68). In particular, was reduced in aged rats, and rescued by probiotic treatment, acting as a gut-targeted antioxidant (69). In this way, disruptions in these four metabolites may play a role in ELA-related brain network alternations that are mediated by oxidative stress pathways and may contribute to clinically meaningful neurophysiological consequences.

An alternative, although potentially related mechanism, is supported by all four metabolites being intimately involved in the metabolism of glutamate and related compounds. Gamma-methyl ester is a metabolite of glutamate (70), while 5-oxoproline is a precursor and closely-related analogue of glutamate (68). Furthermore, 5-oxoproline plays a critical role in glutamate clearance, by stimulating glutamate transport from the brain, and inhibiting its uptake by endothelial cells of the blood-brain barrier (71). The observed reduction in 5-oxoproline may therefore interfere with CNS clearance of glutamate, which at increased concentrations can be particularly excitotoxic (72) in those with a history of high ELA. Additionally, a role for urate-induced, astrocyte-mediate protection against excitotoxicity has been reported in vitro (73). Our findings suggest that a reduction in these metabolites may lower the threshold for cytotoxicity while simultaneously increasing CNS concentrations of glutamate, thereby increasing the risk for excitotoxity and cell death.
Early Life Adversity is Associated with Brain Functional Connectivity

Many types of ELA have been reported to alter brain structure and connectivity, including amygdala, prefrontal, limbic, hippocampal, and striatal regions (19, 20, 74). Here, we identify additional brain regions whose connectivity was significantly correlated with greater ELA scores, which may explain the relationship with psychological outcomes later in life. In particular, we report reduced connectivity of the precuneus, a default mode network region critical for aspects of social cognition (75), and self-consciousness and interpretation (76), which may point to altered evaluation of self and others underlying anxious feelings. Indeed, default mode efficiency is negatively correlated with anxiety in young adults (77, 78), and default mode connectivity relates to responsiveness during anxiety learning (77, 78) as well as being heavily implicated in depressive symptoms (79). Additionally, our findings of decreased connectivity involving emotion regulation networks such as the anterior cingulate cortex, which is involved in conflict monitoring (80) and emotional and cognitive attention (81), and increased connectivity of the insula, a key region in the salience network (82), may suggest modified ability to regulate emotional responses. We report increased connectivity of frontal and parietal sensorimotor regions, and central executive and autonomic areas, which are consistent with a meta-analysis implicating executive control, salience, and sensorimotor networks in anxiety (83).

The fact that ELA disrupts many regions involved in cognitive and emotional processes, which are highly vulnerable to persistent deleterious effects of ELA (84), may present a mechanism underlying our finding that early adversity correlates with later stress and anxiety. Similarly, measures of centrality and segregation in brain regions implicated in emotion and salience reportedly correlate with ELA (15), suggesting that these regions may underly current psychological manifestations of early trauma. This potential mechanism is further supported by findings that functional connectivity of regions including amygdala, putamen, and middle frontal gyms, as well as regions we also identified such as middle temporal and superior frontal gyri differentiated patients with generalized anxiety from healthy controls (85).

A History of Early Life Adversity Correlates with Alternatives in Brain-Gut-Microbiome Axis and Current Psychiatric Symptinns

We also identified significant relationships between fecal metabolites and altered functional brain connectivity measures involving, most notably, the sensorimotor and default mode networks, which have been implicated in both anxiety (77, 78, 83) and depression (79). Previous work has underscored robust relationships between the BGM and psychological outcomes across the lifetime. Probiotic treatment in healthy adults was sufficient to reduce resting state connectivity in somatosensory and insular areas during an emotional attention task (86), and to increase prefrontal cortex activity and reduce baseline and induced stress (87). Connectivity of reward regions has been related to microbiome-derived indole metabolites and anxiety and food addiction outcomes in adults (88), and connectivity of regions involved in salience, emotion regulation, and sensorimotor function correlated with microbial diversity and cognitive outcomes in infants (89).

Gut microbial metabolites may influence brain network connectivity through both direct and indirect mechanisms. While 5-oxoproline decreases entry of amino acids into the brain by acting on transporters (90), urate is capable of passing across the blood-brain barrier and acts as a pro-inflammatory agent (91). However, since our metabolites are measured in feces rather than serum, we cannot say with certainty whether these metabolites have any direct effects on the brain. Alternatively, these metabolites may act indirectly via vagal afferent nerve pathways (92), (93), resulting in altered vagal signaling due to metabolite-induced oxidative stress and excitoxicity which in turn may lead to the observed changes in functional connectivity.

Current stress and other negative emotional states also interact with the BGM axis. We present this in the current study, with PSS and HAD anxiety and depression scores correlating significantly with urate, malate, glutamate, gamma-methyl ester, and 5-oxoproline, as well as with brain functional connectivity of sensorimotor-central executive and default mode-central autonomic regions. These relationships are of significance due to the potential functional influence of altered sensory modalities and orbitofrontal cortex function, which is critical for decision-making (94), on negative psychological states. Similar findings have been reported in the context of food addiction, with amygdala circuitry and the exclusively gut microbiota-derived indole metabolite skatole correlating with higher food addiction scores (88). Additionally, stress-based disorders such as PTSD have been related to altered connectivity in the hippocampus (95) as well as in amygdala-insula circuits (96). Acute stress has also been related to metabolites, with increased CSF homovanillic acid correlating with induced symptoms in PTSD patients (97). However, these past studies do not separate out contributions of past adversity from current experiences of stress and anxiety.

### Clinical Implications and Conclusions

Our findings provide evidence to support the hypothesis that traumatic experiences during critical periods of brain and microbiome development can shape long-term changes in BGM interactions. We suggest that this occurs via the effect of ELA on central autonomic networks and on autonomic nervous system output to alter gut microbial function. The observed dysregulation of glutamate pathways may result in excitotoxicity and oxidative stress, disrupting neural circuit assembly and existing brain network connectivity, and increasing the risk of anxiety and depression.

### REFERENCES

1. Tomalski P, Johnson M H (2010): The effects of early adversity on the adult and developing brain. Curr Opin Psychiatry. 23:233-238.
2. Shonkoff J P, Garner A S, Committee on Psychosocial Aspects of C, Family H, Committee on Early Childhood A, Dependent C, et al. (2012): The lifelong effects of early childhood adversity and toxic stress. Pediatrics. 129:e232-246.
3. Romens S E, McDonald J, Svaren J, Pollak S D (2015): Associations between early life stress and gene methylation in children. Child Dev. 86:303-309.
4. Carpenter L L, Gawuga C E, Tyrka A R, Lee J K, Anderson G M, Price L H (2010): Association between plasma IL-6 response to acute stress and early-life adversity in healthy adults. Neuropsychopharmacology. 35:2617-2623.
5. Joung K E, Park K H, Zaichenko L, Sahin-Efe A, Thakkar B, Brinkoetter M, et al. (2014): Early life adversity is associated with elevated levels of circulating leptin, irisin, and decreased levels of adiponectin in midlife adults. J Clin Endocrinol Metab. 99:E1055-1060.
6. Foster J A, Rinaman L, Cryan J F (2017): Stress & the gut-brain axis: Regulation by the microbiome. Neurobiol Stress. 7:124-136.
7. Mayer E A (2011): Gut feelings: the emerging biology of gut—brain communication. Nature Reviews Neuroscience. 12:453-466.
8. Osadchiy V, Mayer, E. A., Bhatt, R., Labus, J. S., Gao, L., Kilpatrick, L. A., Liu, C., Tillisch, K., Naliboff, B., Chang, L., Gupta, A. (2019): History of early life adversity is associated with increased food addiction and sex-specific alterations in reward network connectivity in obesity. Obesity Science & Practice. 5:416-436.
9. Gupta A, Labus J, Kilpatrick L A, Bonyadi M, Ashe-McNalley C, Heendeniya N, et al. (2016): Interactions of early adversity with stress-related gene polymorphisms impact regional brain structure in females. Brain structure & function. 221:1667-1679.
10. Labus J S, Osadchiy V, Hsiao E Y, Tap J, Derrien M, Gupta A, et al. (2019): Evidence for an association of gut microbial Clostridia with brain functional connectivity and gastrointestinal sensorimotor function in patients with irritable bowel syndrome, based on tripartite network analysis. Microbiome. 7:45.
11. Gupta A, Kilpatrick L, Labus J, Tillisch K, Braun A, Hong J Y, et al. (2014): Early adverse life events and resting state neural networks in patients with chronic abdominal pain: evidence for sex differences. Psychosom Med. 76:404-412.
12. Berman S, Suyenobu B, Naliboff BD, Bueller J, Stains J, Wong H, et al. (2012): Evidence for alterations in central noradrenergic signaling in irritable bowel syndrome. NeuroImage. 63:1854-1863.
13. Levine M E, Cole, S. W., Weir, D. R., Crimmins, E. M. (2015): Childhood and later life stressors and increased inflammatory gene expression at older ages. Social Science & Medicine. 130:16-22.
14. Teicher M H, Samson, J. A., Anderson, C. M., Ohashi, K. (2016): The effects of childhood maltreatment on brain structure, function and connectivity. Nature Reviews. 17:652-666.
15. Gupta A, Mayer E A, Acosta J R, Hamadani K, Torgerson C, van Horn J D, et al. (2017): Early adverse life events are associated with altered brain network architecture in a sex-dependent manner. Neurobiol Stress. 7:16-26.
16. Etkin A, Egner, T., Peraza, D. M., Kandel, E. R., Hirsch, J. (2006): Resolving Emotional Conflict: A Role for the Rostral Anterior Cingulate Cortex in Modulating Activity in the Amygdala. Neuron. 51:871-882.
17. Cisler J M, James, G. A., Tripathi, S., Mletzko, T., Heim, C., Hu, X. P., Mayberg, H. S., Nemeroff, C. B., Kilts, C. D. (2013): Differential functional connectivity within an emotion regulation neural network among individuals resilient and susceptible to the depressogenic effects of early life stress. Psychological Medicine. 43:507-518.
18. Stein M B, Simmons, A. N., Feinstein, J. S., Paulus, M. P. (2007): Increased amygdala and insula activation during emotion processing in anxiety-prone subjects. The American Journal of Psychiatry. 164:318-327.
19. Hodel A S, Hunt R H, Cowell R A, Van Den Heuvel S E, Gunnar M R, Thomas K M (2015): Duration of early adversity and structural brain development in post-institutionalized adolescents. Neuroimage. 105:112-119.
20. Miskovic V, Schmidt L A, Georgiades K, Boyle M, Macmillan H L (2010): Adolescent females exposed to child maltreatment exhibit atypical EEG coherence and psychiatric impairment: linking early adversity, the brain, and psychopathology. Dev Psychopathol. 22:419-432.
21. Honeycutt J A, Demaestri C, Peterzell S, Silveri M M, Cai X, Kulkarni P, et al. (2020): Altered corticolimbic connectivity reveals sex-specific adolescent outcomes in a rat model of early life adversity. Elife. 9.
22. Zijlmans M A, Korpela K, Riksen-Walraven J M, de Vos W M, de Weerth C (2015): Maternal prenatal stress is associated with the infant intestinal microbiota. Psychoneuroendocrinology. 53:233-245.
23. Yassour M, Vatanen T, Silj ander H, Hamalainen A M, Harkonen T, Ryhanen S J, et al. (2016): Natural history of the infant gut microbiome and impact of antibiotic treatment on bacterial strain diversity and stability. Sci Transl Med. 8:343ra381.
24. Backhed F, Roswall J, Peng Y, Feng Q, Jia H, Kovatcheva-Datchary P, et al. (2015): Dynamics and Stabilization of the Human Gut Microbiome during the First Year of Life. Cell Host Microbe. 17:852.
25. Bergstrom A, Skov T H, Bahl M I, Roager HM, Christensen L B, Ejlerskov K T, et al. (2014): Establishment of intestinal microbiota during early life: a longitudinal, explorative study of a large cohort of Danish infants. Appl Environ Microbiol. 80:2889-2900.
26. De Palma G, Blennerhassett P, Lu J, Deng Y, Park A J, Green W, et al. (2015): Microbiota and host determinants of behavioural phenotype in maternally separated mice. Nat Commun. 6:7735.
27. Moussaoui N, Jacobs, J. P., Larauche, M., Biraud, M., Million, M., Mayer, E., Tache, Y. (2017): Chronic Early-life Stress in Rat Pups Alters Basal Corticosterone, Intestinal Permeability, and Fecal Microbiota at Weaning: Influence of Sex. Journal of Neurogastroenterology and Motility. 23:135-143.
28. Dinan T G, Cryan J F (2012): Regulation of the stress response by the gut microbiota: implications for psychoneuroendocrinology. Psychoneuroendocrinology. 37:1369-1378.
29. Bharwani A, Mian M F, Foster J A, Surette M G, Bienenstock J, Forsythe P (2016): Structural & functional consequences of chronic psychosocial stress on the microbiome & host. Psychoneuroendocrinology. 63:217-227.
30. Neufeld K M, Kang N, Bienenstock J, Foster J A (2011): Reduced anxiety-like behavior and central neurochemical change in germ-free mice. Neurogastroenterol Motil. 23:255-264, e119.
31. Yatsunenko T, Rey F E, Manary M J, Trehan I, Dominguez-Bello M G, Contreras M, et al. (2012): Human gut microbiome viewed across age and geography. Nature. 486:222-227.
32. Wall R, Cryan S F, Ross R P, Fitzgerald G F, Dinan T G, Stanton C (2014): Bacterial neuroactive compounds produced by psychobiotics. Adv Exp Med Biol. 817:221-239.
33. Melander A, Olsson J, Lindberg G, Salzman A, Howard T, Stang P, et al. (1999): 35th Annual Meeting of the European Association for the Study of Diabetes: Brussels, Belgium, 28 Sep.-2 Oct. 1999. Diabetologia. 42:A1-A330.
34. Roy A (1999): CSF 5-HIAA correlates with neuroticism in depressed patients. J Affect Disord. 52:247-249.
35. Zheng P, Zeng B, Zhou C, Liu M, Fang Z, Xu X, et al. (2016): Gut microbiome remodeling induces depressive-like behaviors through a pathway mediated by the host's metabolism. Mol Psychiatry. 21:786-796.
36. Veenema A H, Blume A, Niederle D, Buwalda B, Neumann I D (2006): Effects of early life stress on adult male aggression and hypothalamic vasopressin and serotonin. Eur J Neurosci. 24:1711-1720.
37. Dalile B, Vervliet, B., Bergonzelli, G., Verbeke, K., Van Oudenhove, L. (2020): Colon-delivered short-chain fatty acids attenuate the cortisol response to psychosocial stress in healthy men: a randomized, placebo-controlled trial. Neuropsychopharmacology. 0:1-10.
38. Van de Wouw M, Boehme, M., Lyte, J. M., Wiley, N., Strain, C., O'Sullivan, O., Clarke, G., Stanton, C., Dinan, T. G., Cryan, J. F. (2018): Short-chain fatty acids: microbial metabolites that alleviate stress-induced brain-gut axis alterations. The Journal of Physiology. 596:4923-4944.
39. Lyte M, Vulchanova L, Brown D R (2011): Stress at the intestinal surface: catecholamines and mucosa-bacteria interactions. Cell Tissue Res. 343:23-32.
40. Bremner J D, Bolus R, Mayer E A (2005): The early trauma inventory self report (ETI-SR). Gastroenterology. 128:A340-A340.
41. Bremner J D, Bolus R, Mayer E A (2007): Psychometric properties of the Early Trauma Inventory-Self Report. Journal of Nervous and Mental Disease. 195:211-218.
42. Cohen S, Kamarck T, Mermelstein R (1983): A global measure of perceived stress. J Health Soc Behay. 24:385-396.
43. Zigmond A S, Snaith R P (1983): The hospital anxiety and depression scale. Acta psychiatrica Scandinavica. 67:361-370.
44. Kroenke K, Spitzer R L, Williams J B (2002): The PHQ-15: validity of a new measure for evaluating the severity of somatic symptoms. Psychosom Med. 64:258-266.
45. Evans A M, DeHaven C D, Barrett T, Mitchell M, Milgram E (2009): Integrated, nontargeted ultrahigh performance liquid chromatography/electrospray ionization tandem mass spectrometry platform for the identification and relative quantification of the small-molecule complement of biological systems. Anal Chem. 81:6656-6667.
46. Xia J, Sinelnikov I V, Han B, Wishart DS (2015): MetaboAnalyst 3.0—making metabolomics more meaningful. Nucleic Acids Res. 43:W251-257.
47. Labus J S, Van Horn J D, Gupta A, Alaverdyan M, Torgerson C, Ashe-McNalley C, et al. (2015): Multivariate morphological brain signatures predict patients with chronic abdominal pain from healthy control subjects. Pain. 156:1545-1554.
48. Labus J S, Naliboff B, Kilpatrick L, Liu C, Ashe-McNalley C, Dos Santos I R, et al. (2015): Pain and Interoception Imaging Network (PAIN): A multimodal, multisite, brain-imaging repository for chronic somatic and visceral pain disorders. Neuroimage.
49. Gupta A, Mayer E A, Sanmiguel C P, Van Horn J D, Woodworth D, Ellingson B M, et al. (2015): Patterns of brain structural connectivity differentiate normal weight from overweight subjects. Neuroimage Clin. 7:506-517.
50. Gupta A, Mayer E A, Labus J S, Bhatt R R, Ju T, Love A, et al. (2018): Sex Commonalities and Differences in Obesity-Related Alterations in Intrinsic Brain Activity and Connectivity. Obesity (Silver Spring). 26:340-350.
51. Gupta A, Mayer E A, Hamadani K, Bhatt R, Fling C, Alaverdyan M, et al. (2017): Sex differences in the influence of body mass index on anatomical architecture of brain networks. Int J Obes (Loud). 41:1185-1195.
52. Carrola J, Rocha C M, Barros A S, Gil A M, Goodfellow B J, Carreira I M, et al. (2011): Metabolic signatures of lung cancer in biofluids: NMR-based metabonomics of urine. J Proteome Res. 10:221-230.
53. Kim H J, Kim J H, Noh S, Hur H J, Sung M J, Hwang J T, et al. (2011): Metabolomic analysis of livers and serum from high-fat diet induced obese mice. J Proteome Res. 10:722-731.
54. Benjamini Y, Hochberg Y (1995): Controlling the false discovery rate: a practical and powerful approach to multiple testing. Journal of the Royal Statistical Society Series B (Methodological). 57:289-300.
55. Ilievski V, Kinchen JM, Prabhu R, Rim F, Leoni L, Unterman TG, et al. (2016): Experimental Periodontitis Results in Prediabetes and Metabolic Alterations in Brain, Liver and Heart: Global Untargeted Metabolomic Analyses. J Oral Biol (Northborough). 3.
56. Donohoe D R, Garge N, Zhang X, Sun W, O'Connell T M, Bunger M K, et al. (2011): The microbiome and butyrate regulate energy metabolism and autophagy in the mammalian colon. Cell Metab. 13:517-526.
57. Scheperjans F, Pekkonen E, Kaakkola S, Auvinen P (2015): Linking Smoking, Coffee, Urate, and Parkinson's Disease—A Role for Gut Microbiota? J Parkinsons Dis. 5:255-262.
58. Behr C, Kamp H, Fabian E, Krennrich G, Mellert W, Peter E, et al. (2017): Gut microbiome-related metabolic changes in plasma of antibiotic-treated rats. Arch Toxicol. 91:3439-3454.
59. Li B, Guo K, Zeng L, Zeng B, Huo R, Luo Y, et al. (2018): Metabolite identification in fecal microbiota transplantation mouse livers and combined proteomics with chronic unpredictive mild stress mouse livers. Transl Psychiatry. 8:34.
60. Weisskopf M G, O'Reilly E, Chen H, Schwarzschild M A, Ascherio A (2007): Plasma urate and risk of Parkinson's disease. Am J Epidemiol. 166:561-567.
61. Schiavone S, Colaianna M, Curtis L (2015): Impact of early life stress on the pathogenesis of mental disorders: relation to brain oxidative stress. Curr Pharm Des. 21:1404-1412.
62. Epel E S, Blackburn E H, Lin J, Dhabhar F S, Adler N E, Morrow J D, et al. (2004): Accelerated telomere shortening in response to life stress. Proc Natl Acad Sci USA. 101:17312-17315.
63. Tyrka A R, Parade S H, Price L H, Kao H T, Porton B, Philip N S, et al. (2016): Alterations of Mitochondrial DNA Copy Number and Telomere Length With Early Adversity and Psychopathology. Biol Psychiatry. 79:78-86.
64. Tyrka A R, Price L H, Kao H T, Porton B, Marsella S A, Carpenter L L (2010): Childhood maltreatment and telomere shortening: preliminary support for an effect of early stress on cellular aging. Biol Psychiatry. 67:531-534.
65. Ames B N, Cathcart, R., Schwiers, E., Hochstein, P (1981): Uric acid provides an antioxidant defense in humans against oxidant- and radical-caused aging and cancer: a hypothesis. . Proceedings of the National Academy of Sciences. 78:6858-6862.
66. Wu J L, Wu Q P, Yang X F, Wei M K, Zhang J M, Huang Q, et al. (2008): L-malate reverses oxidative stress and antioxidative defenses in liver and heart of aged rats. Physiol Res. 57:261-268.
67. Randhawa M, Sangar V, Tucker-Samaras S, Southall M (2014): Metabolic signature of sun exposed skin suggests catabolic pathway overweighs anabolic pathway. PLoS One. 9:e90367.
68. Kumar A B, A. K. (2012): Pyroglutamic acid: throwing light on a lightly studied metabolite. Current Science. 102:288-297.
69. Hor Y Y, Lew L C, Jaafar M H, Lau A S, Ong J S, Kato T, et al. (2019): Lactobacillus sp. improved microbiota and metabolite profiles of aging rats. Pharmacol Res. 146:104312.
70. Tsuge Y K, A. (2017): Production of amino acids (L-Glutamic Acid and L-Lysine) from biomass. In: Fang Z, Smith, Jr. R., Qi, X., editor. Production of Platform Chemicals from Sustainable Resources: Springer, Singapore, pp 437-455.
71. Hawkins R A, Simpson, I. A., Mokashi, A., Vina, J. R. (2006): Pyroglutamate stimulates Na+-dependent glutamate transport across the blood brain barrier.FEBS Letters. 580:4382-4386.
72. Dong X, Wang, Y., Qin, Z. (2009): Molecular mechanisms of excitoxocitiy and their relevance to pathogenesis of neurodegenerative diseases. Acta Pharmacologica Sinica. 30:379-387.
73. Du Y, Chen, C. P., Tseng, C., Eisenberg, Y., Firestein, B. L. (2007): Astroglia-mediated effects of uric acid to protect spinal cord neurons from glutamate toxicity. Glia. 55:463-472.
74. Gee D G, Gabard-Durnam L J, Flannery J, Goff B, Humphreys K L, Telzer E H, et al. (2013): Early developmental emergence of human amygdala-prefrontal connectivity after maternal deprivation. Proc Nall Acad Sci USA. 110:15638-15643.
75. Li W, Mai, X., Liu, C. (2014): The default mode network and social understanding of others: what do brain connectivity studies tell us. Frontiers in Human Neuroscience. 8.
76. Cavanna A E, Trimble M R (2006): The precuneus: a review of its functional anatomy and behavioural correlates. Brain. 129:564-583.
77. Zidda F, Andoh, J., Pohlack, S., Winkelmann, T., Dinu-Biringer, R., Cavalli, J., Ruttorf, M., Nees, F., Flor, H. (2017): Default mode network connectivity of fear- and anxiety-related cue and context conditioning. Neuroimage. 165:190-199.
78. Tao Y, Liu, B., Zhang, X., Li, J., Qin, W., Yu, C., Jiang, T. (2015): The structural connectivity pattern of the default mode network and its association with memory and anxiety. Frontiers in Neuronatomy. 9.
79. Brakowski J, Spinelli, S., Dorig, N., Bosch, O. G., Manoliu, A., Holtforth, M. G., Seifritz, E. (2017): Resting state brain network function in major depression—Depression symptomatology, antidepressant treatment effects, future research. Journal of Psychiatric Research. 92:147-159.
80. Kerns J G, Cohen J D, MacDonald A W, 3rd, Cho R Y, Stenger V A, Carter C S (2004): Anterior cingulate conflict monitoring and adjustments in control. Science. 303:1023-1026.
81. Bush G, Luu P, Posner M I (2000): Cognitive and emotional influences in anterior cingulate cortex. Trends in cognitive sciences. 4:215-222.
82. Menon V, Uddin L Q (2010): Saliency, switching, attention and control: a network model of insula function. Brain Struct Funct. 214:655-667.
83. Xu J, Van Dam, N.T., Feng, C., Luo, Y., Ai, H., Gu, R., Xu, P. (2019): Anxious brain networks: A coordinate-based activation likelihood estimation meta-analysis of resting-state functional connectivity studies in anxiety. Neuroscience & Biobehavioral Reviews. 96:21-30.
84. Pechtel P, Pizzagalli, D. A. (2011): Effects of early life stress on cognitive and affective function: an integrated review of human literature. Psychopharmacology. 214:55-70.
85. Qiao J, Li, A., Cao, C., Wang, Z., Sun, J., Xu, G. (2017): Aberrant Functional Network Connectivity as a Biomarker of Generalized Anxiety Disorder. Frontiers in Human Neuroscience. 11.
86. Tillisch K, Labus, J., Kilpatrick, L., Jiang, Z., Stains, J., Ebrat, B., Guyonnet, D., Legrain-Raspaud, S., Trotin, B., Naliboff, B., Mayer, E. A. (2013): Consumption of fermented milk product with probiotic modulates brain activity. Gastroenterology. 144:1394-1401.
87. Allen A P, Hutch, W., Borre, Y. E., Kennedy, P. J., Temko, A., Boylan, G., Murphy, E., Cryan, J. F., Dinan, T. G., Clarke, G. (2016): Bifidobacterium longum 1714 as a translational psychobiotic: modulation of stress, electrophysiology and neurocognition in healthy volunteers. Translational Psychiatry. 6.
88. Osadchiy V, Labus JS, Gupta A, Jacobs J, Ashe-McNalley C, Hsiao E Y, et al. (2018): Correlation of tryptophan metabolites with connectivity of extended central reward network in healthy subjects. PLoS One. 13:e0201772.
89. Gao W, Salzwedel, A. P., Carlson, A. L., Xia, K., Azcarate-Peril, M. A., Styner, M. A., Thompson, A. L., Geng, X., Goldman, B. D., Gilmore, J. H., Knickmeyer, R. C. (2019): Gut microbiome and brain functional connectivity in infants—a preliminary study focusing on the amygdala. Psychopharmacology. 236:1641-1651.
90. Hawkins R A, O'Kane, R. L., Simpson, I. A., Vina, J. R. (2006): Structure of the blood-brain barrier and its role in the transport of amino acids. The Journal of Nutrition. 136:218S-226S.
91. Shao X, Lu, W., Gao, F., Li, D., Hu, J., Li, Y., Zuo, Z., Jie, H., Zhao, Y., Cen, X. (2016): Uric acid induces cognitive dysfunction through hippocampal inflammation in rodents and humans. Journal of Neuroscience. 36:10990-11005.
92. Bartolomei F, Bonini, F., Vidal, E., Trebuchon, A., Lagarde, S., Lambert, I., McGonigal, A., Scavarda, D., Carron, R., Benar, C.G. (2016): How does vagal nerve stimulation (VNS) change EEG brain functional connectivity? Epilepsy Research. 126:141-146.
93. Cao J, Lu, K., Powley, T.L., Liu, Z. (2017): Vagal nerve stimulation triggers widespread responses and alters large-scale functional connectivity in the rat brain. PLoS One. 12:e0189518.
94. Bechara A, Damasio H, Damasio A R (2000): Emotion, decision making and the orbitofrontal cortex. Cereb Cortex. 10:295-307.
95. Chen A C, Etkin A (2013): Hippocampal network connectivity and activation differentiates post-traumatic stress disorder from generalized anxiety disorder. Neuropsychopharmacology. 38:1889-1898.
96. Rabinak C A, Angstadt M, Welsh R C, Kenndy A E, Lyubkin M, Martis B, et al. (2011): Altered amygdala resting-state functional connectivity in post-traumatic stress disorder. Front Psychiatry. 2:62.
97. Geracioti T D, Jr., Jefferson-Wilson L, Strawn J R, Baker D G, Dashevsky B A, Horn P S, et al. (2013): Effect of traumatic imagery on cerebrospinal fluid dopamine and serotonin metabolites in posttraumatic stress disorder. J Psychiatr Res. 47:995-998.
98. Tong M, Jacobs J P, McHardy I H, Braun J (2014): Sampling of intestinal microbiota and targeted amplification of bacterial 16S rRNA genes for microbial ecologic analysis. Curr Protoc Immunol. 107:7 41 41-47 41 11.
99. Caporaso J G, Kuczynski J, Stombaugh J, Bittinger K, Bushman F D, Costello E K, et al. (2010): QIIME allows analysis of high-throughput community sequencing data. Nat Methods. 7:335-336.
100. Lozupone C, Knight R (2005): UniFrac: a new phylogenetic method for comparing microbial communities. Appl Environ Microbiol. 71:8228-8235.
101. Anderson M J (2001): A new method for non-parametric multivariate analysis of variance. Austral Ecology. 26:32-46.
102. Love M I, Huber W, Anders S (2014): Moderated estimation of fold change and dispersion for RNA-seq data with DESeq2. Genome Biol. 15:550.
103. Tausch E, Beck P, Schlenk R F, Jebaraj B J, Dolnik A, Yosifov D Y, et al. (2020): Prognostic and predictive role of gene mutations in chronic lymphocytic leukemia: results from the pivotal phase III study COMPLEMENT1. Haematologica.
104. Schaefer A, Kong, R., Gordon, E. M., Laumann, T. O., Zuo, X., Holmes, A. J., Eickhoff, S. B., Yeo, B. T. T. (2018): Local-Global Parcellation of the Human Cerebral Cortex from Intrinsic Functional Connectivity MRI. Cerebral Cortex. 28:3095-3114.
105. Desikan R S, Segonne F, Fischl B, Quinn B T, Dickerson B C, Blacker D, et al. (2006): An automated labeling system for subdividing the human cerebral cortex on MRI scans into gyral based regions of interest. Neuroimage. 31:968-980.
106. Destrieux C, Fischl B, Dale A, Halgren E (2010): Automatic parcellation of human cortical gyri and sulci using standard anatomical nomenclature. Neuroimage. 53:1-15.
107. Desikan R S, Segonne, F., Fischl, B., Quinn, B. T., Dickerson, B. C., Blacker, D., Buckner, R. L., Dale, A. M., Maguire, R. P., Hyman, B. T., Albert, M. S., Killiany, R. J. (2006): An automated labeling system for subdividing the human cerebral cortex on MRI scans into gyral based regions of interest. NeuroImage. 31:968-980.
108. Edlow B L, Takahashi, E., Wu, O., Benner, T., Dai, G., Bu, L., Grant, P. E., Greer, D. M., Greenberg, S. M., Kinney, H. C., Folkerth, R. D. (2012): Neuroanatomic connectivity of the human ascending arousal system critical to consciousness and its disorders. J Neuropathol Exp Neurol. 71:531-546.
109. Irimia A, Van Horn J D (2012): The structural, connectomic and network covariance of the human brain. Neuroimage. 66C:489-499.
110. Whitfield-Gabrieli S, Nieto-Castanon, A. (2012): Conn: A Functional Connectivity Toolbox for Correlated and Anticorrelated Brain Networks. Brain Connect. 2:125-141.
111. Behzadi Y, Restom, K., Liau, J., Liu, T. T. (2007): A Component Based Noise Correction Method (CompCor) for BOLD and Perfusion Based fMRI. Neuroimage. 37 :90-101.
112. Ciric R, Wolf D H, Power J D, Roalf D R, Baum G L, Ruparel K, et al. (2017): Benchmarking of participant-level confound regression strategies for the control of motion artifact in studies of functional connectivity. Neuroimage. 154:174-187.
113. Makris N, Goldstein, J. M., Kenendy, D., Hodge, S. M., Caviness, V. S., Faraone, S. V., Tsuang, M. T., Seidman, L. J. (2006): Decreased volume of left and total anterior insular lobule in schizophrenia. Schizophrrenia Research. 83:155-171.
114. Frazier J A, Chiu, S., Breeze, J. L., Makris, N., Lange, N., Kennedy, D. N., Herbert, M. R., Bent, E. K., Koneru, V. K., Dieterich, M. E., Hodge, S. M., Rauch, S. L., Grant, P. E., Cohen, B. M., Seidman, L. J., Caviness, V. S., Bierdman, J. (2005): Structural Brain Magnetic Resonance Imaging of Limbic and Thalamic Volumes in Pediatric Bipolar Disorder. The American Journal of Psychiatry. 162:1256-1265.
115. Goldstein J M, Seidman, L. J., Makris, N., Ahern, T., O'Brien, L. M., Caviness, V. S. Jr., Kennedy, D. N., Faraone, S. V., Tsuang, M. T. (2007): Hypothalamic abnormalities in schizophrenia: sex effects and genetic vulnerability. Biological Psychiatry. 61:935-945.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method of identifying a subject with a history of early-life adversity (ELA), comprising:
(a) measuring the level of one or more metabolites selected from glutamate, gamma-methyl ester, 5-oxoproline, malate, lithocholic acid sulfate, and urate in a sample obtained from the subject;
(b) comparing the level detected from the step (a) to a normal level of the metabolite;
wherein a decreased level of the metabolite in the subject sample indicates that the subject has a history of ELA.

2. The method of claim 1, wherein the method further comprises obtaining a sample from the subject prior to step (a).

3. The method of claim 2, wherein the level of the metabolite is measured by mass spectrometry, HPLC, or NMR.

4. The method of any one of claims 1-3, wherein the level of the metabolite is assessed by liquid-chromatography-tandem mass spectrometry.

5. The method of any one of claims 1-4, wherein the normal level of the metabolite is a reference value, e.g., representative of levels measured in a number of control samples.

6. The method of any one of claims 1-5, wherein the normal level of the metabolite is determined from a control sample.

7. The method of any one of claims 1-6, wherin the control sample is obtained from a subject with an Early Traumatic Inventory-Self Report (ETI-SR) total score ≤4.

8. The method of any one of claims 1-7, wherein the control sample is obtained from a subject without a history of ELA.

9. The method of any one of claims 1-8, wherein the sample is selected from organs, tissue, body fluids and cells.

10. The method of any one of claims 1-9, wherein the body fluid is whole blood, serum, plasma, sputum, spinal fluid, lymph fluid, skin secretions, respiratory secrections, intestinal secretions, genitourninary tract secretions, tears, milk, buccal scrape, saliva, cerebrospinal fluid, urine, or stool.

11. The method of any one of claims 1-10, wherein the sample is a stool sample.

12. The method of any one of claims 1-11, wherein the level of the metabolite in the subject sample is equal to, or less than half of the normal level.

13. The method of any one of claims 1-12, wherein the subject to be evaluated has an ETI-SR total score >4.

14. A method of preventing, treating, or reducing psychological distress in a subject with a history of ELA, comprising administering to the subject an agent that increases the level and/or activity of at least one metabolite selected from glutamate, gamma-methyl ester, 5-oxoproline, malate, lithocholic acid sulfate, and urate.

15. The method of claim 14, wherein the agent is glutamate, gamma-methyl ester, 5-oxoproline, malate, lithocholic acid sulfate, or urate.

16. The method of claim 14, wherein the agent is a analogue, a prodrug, or a pharmaceutically acceptable salt of glutamate, gamma-methyl ester, 5-oxoproline, malate, lithocholic acid sulfate, or urate.

17. The method of claim 14, wherein the agent is a probiotic supplement.

18. The method of claim 17, wherein the probiotic supplement comprises Lactobacillus plantarum, or microbiota enriched in Prevotella.

19. The method of any one of claims 14-18, wherein the psychological distress is selected from depression, anxiety, stress, and negative mood.

20. The method of any one of claims 14-19, wherein the method further comprises identifying a subject with a history of early-life adversity (ELA) according to claims 1-13 prior to administering the agent to such subject.

21. The method of any one of claims 14-20, wherein the agent is administered in a pharmaceutically acceptable formulation.

22. A method for assessing the progression of psychological distress in a subject with a history of ELA, the method comprising:
(a) detecting in a subject sample at a first point in time the level of one or more metabolites selected from glutamate, gamma-methyl ester, 5-oxoproline, malate, lithocholic acid sulfate, and urate;
(b) repeating step (a) at a subsequent point in time; and
(c) comparing the level detected in steps (a) and (b), and therefrom assessing the progression of psychological distress in the subject.

23. A method of assessing the efficacy of an agent for treating or reducing psychological distress in a subject with a history of ELA, the method comprising:
(a) detecting in a subject sample at a first point in time the level of one or more metabolites selected from glutamate, gamma-methyl ester, 5-oxoproline, malate, lithocholic acid sulfate, and urate;
(b) administering the agent to the subject;
(c) repeating step (a) during at least one subsequent point in time after administration of the agent; and
(d) comparing the level of the one or more metabolites from steps (a) and (c),
wherein a significantly increased level of one or more metabolites selected from glutamate, gamma-methyl ester, 5-oxoproline, malate, lithocholic acid sulfate, and urate in the subsequent sample, relative to the sample at the first point in time, indicates that the agent treats psychological distress in the subject.

24. The method of claim 22 or 23, wherein between the first point in time and the subsequent point in time, the subject has undergone treatment, completed treatment, and/or is in remission for psychological distress.

25. The method of any one of claims 22-24, wherein the first and/or at least one subsequent sample is selected from ex vivo and in vivo samples.

26. The method of any one of claims 22-25, wherein the first and/or at least one subsequent sample is obtained from an animal model of ELA.

27. The method of any one of claims 22-26, wherein the first and/or at least one subsequent sample is a portion of a single sample or pooled samples obtained from the subject.

28. The method of any one of claims 22-27, wherein the sample comprises cells, cell lines, histological slides, paraffin embedded tissue, fresh frozen tissue, fresh tissue, biopsies, whole blood, serum, plasma, sputum, spinal fluid, lymph fluid, skin secretions, respiratory secrections, intestinal secretions, genitourninary tract secretions, tears, milk, buccal scrape, saliva, cerebrospinal fluid, urine, and stool obtained from the subject.

29. The method of any one of claims 22-28, wherein the sample is a stool sample.

30. The method of any one of claims 22-29, wherein the psychological distress is selected from depression, anxiety, stress, and negative mood.

31. The method of any one of claims 1-30, wherein the subject is an animal model of ELA.

32. The method of claim 31, wherein the animal model is a rodent model.

33. The method of any one of claims 1-32, wherein the subject is a mammal.

34. The method of any one of claims 1-33, wherein the mammal is a mouse or a human. The method of any one of claims 1-34, wherein the mammal is a human.

36. A kit for assessing whether a subject has a history of ELA, the kit comprising a reagent for assessing the level of one or more metabolites selected from glutamate, gamma-methyl ester, 5-oxoproline, malate, lithocholic acid sulfate, and urate.
